(19) United States
(12) Patent Application Publication
(10) Pub. No.: US 2002/0187564 A1
(43) Pub. Date: Dec. 12, 2002

(54) Microfluidic library analysis

(75) Inventors: Calvin Y.H. Chow, Portola Valley, CA (US); J. Wallace Parce, Palo Alto, CA (US)

Correspondence Address: CALIPER TECHNOLOGIES CORP, 605 FAIRCHILD DRIVE, MOUNTAIN VIEW, CA, 94043, US

(73) Assignee: Caliper Technologies Corp., Mountain View, CA

(21) Appl. No.: 10/159,606

(22) Filed: May 31, 2002

Related U.S. Application Data
(60) Provisional application No. 60/297,022, filed on Jun. 8, 2001.

(51) Int. Cl. G01N 33/53; C12M 1/34; G01N 33/543

(52) U.S. Cl. 436/518; 435/7.1; 435/287.2

(57) ABSTRACT

The present invention provides novel microfluidic devices and methods for storing, reconstituting and accessing one or more library of assay components within library storage elements in a microfluidic device. In particular, the devices and methods of the invention are useful in screening large libraries of molecules.

### CROSS-REFERENCE TO RELATED APPLICATION

This application claims the benefit of U.S. Provisional Patent Application No. 60/297,022 filed Jun. 8, 2001, which is incorporated herein by reference in its entirety for all purposes.

### BACKGROUND OF THE INVENTION

When carrying out chemical or biochemical analyses, assays, syntheses or preparations, a large number of separate manipulations are performed on the material(s) or component(s) to be assayed, including measuring, aliquotting, transferring, diluting, mixing, separating, detecting, incubating, etc. Microfluidic technology miniaturizes these manipulations and integrates them so that they can be executed within one or a few microfluidic devices. For example, pioneering microfluidic methods of performing biological assays in microfluidic systems have been developed, such as those described by Parce et al., “High Throughput Screening Assay Systems in Microscale Fluidic Devices” U.S. Pat. No. 5,942,443 and Knapp et al., “Closed Loop Biochemical Analyzers” (WO 98/45481).

Of particular interest in many fields of science is the screening of, e.g., numerous compounds, patient samples, or molecules against one another or against, e.g., a particular target molecule, gene, etc., in order to, e.g., test for possible interactions, etc. For example, screening of large libraries of molecules is often utilized in pharmaceutical research to select potential targets for pharmaceuticals useful in disease treatments. “Combinatorial” libraries, composed of a collection of generated compounds, can be screened against a particular receptor to test for the presence of, e.g., possible ligands and to quantify the binding of any possible ligands. Screening large libraries of molecules is also important in the search for differences in nucleic acids, e.g., single nucleotide polymorphisms (SNPs).

Current methods of screening large libraries include such methods as using robotic systems that sample library constituents from multiwell plates. However, applications incorporating library analysis using microfluidic systems provide benefits in terms of, e.g., automatability, reagent consumption, and speed. For example, library analysis using a microfluidic system can be performed on fluid volumes on the order of nanoliters or less. Additionally, microfluidic systems allow for precise computer control of many aspects of reagent manipulation (e.g., flowing, heating, mixing, etc.) as well as data acquisition and analysis.

A welcome addition to the art would be the ability to perform high throughput analysis of large libraries, coupled with minimal use of compounds/reagents and the benefits of compound/reagent storage and accessibility. The current invention describes and provides these and other features by providing new methods and microfluidic devices that meet these, and other, goals.

### SUMMARY OF THE INVENTION

The present invention provides methods, systems, kits, and devices using microfluidics for conducting analysis of libraries of compounds. Compounds (molecules, reagents, etc.) to be screened are deposited in dried or otherwise immobilized form in library storage elements (e.g., in microscale reservoirs or in test-microchannels) of microfluidic chips. Fluid (e.g., buffer) is flowed through a complex of microchannels to the library storage elements, or is deposited within the microscale reservoir, to reconstitute the dried or immobilized compounds. The reconstituted compounds are then optionally assayed with respect to selected test compounds and screened for a relevant response (e.g., fluorescence, etc.) that indicates, e.g., binding, activity, or the like.

In one aspect, the invention comprises a microfluidic device of a plurality of library storage elements fluidly coupled to a plurality of microscale channels. In different embodiments, the library storage elements can be contained within microscale reservoirs and/or test-microchannels. In various aspects, the microscale reservoirs comprise a largest dimension of less than, e.g., about 5 millimeters or less, about 1 millimeter or less, or less than about 500 micrometers, or even less than about 300 micrometers. In other aspects, the number of library storage elements comprises between at least about 10 to about 1,000,000 or more, between at least about 100 to at least about 100,000 or more, between at least about 1,000 to at least about 10,000 or more, or between about at least about 60,000 to about 600,000 or more library storage elements. Additionally, in other aspects, the density of library storage elements in the microfluidic device can be from about 5 to about 10,000 library storage elements per square centimeter, from about 100 to about 5,000 library storage elements per square centimeter, from about 1,000 to about 2,500 library storage elements per square centimeter, from about 100 to about 500 library storage elements per square centimeter, or from about 400 to about 4,000 library storage elements per square centimeter. Optionally, the microscale reservoirs can be disposed within a surface of the microfluidic device or, more preferably, the microscale reservoirs can be disposed within an upper surface of the microfluidic device. Additionally, at least one member of the plurality of the library storage elements of the invention comprises a dried or immobilized test compound. Optionally, substantially all members of the plurality of library storage elements comprise a different dried or immobilized test compound. Alternatively, the library storage elements of the microfluidic system comprise dried or immobilized test compounds which are not all substantially different compounds. Furthermore, the plurality of library storage elements can optionally comprise a library of test compounds. At least one member, or substantially all members, of the plurality of microscale channels of the microfluidic device optionally contains a fluidic material, which fluidic material can optionally comprise a buffer.

In one aspect, the current invention comprises a microfluidic system comprising a body structure with a plurality of microscale channels and a plurality of library storage elements along with a fluid delivery system that delivers a portion of fluid to one or more library storage element during operation. In different embodiments, the library storage elements can be contained within microscale reservoirs and/or test-microchannels. Optionally, the microscale reservoirs of the microfluidic system can be less than about 5 millimeters in size, less than about 1 millimeter, less than about 500 micrometers in size, or less than about 300 micrometers in size. Furthermore the microfluidic system can have a plurality of between at least about 10 to at least about 1,000,000 or more library storage elements, between at least about 100 to at least about 100,000 or more library storage elements, between at least about 1,000 to at least about 10,000 or more library storage elements, or between about at least 60,000 to about 600,000 or more library storage elements. Additionally, in other aspects, the density of library storage elements in the microfluidic system can be from about 5 to about 10,000 library storage elements per square centimeter, from about 100 to about 5,000 library storage elements per square centimeter, from about 1,000 to about 2,500 library storage elements per square centimeter, from about 100 to about 500 library storage elements per square centimeter, or from about 400 to about 4,000 or more library storage elements per square centimeter. Optionally, the microscale reservoirs can be disposed within a surface of the body structure of the microfluidic system or, more preferably, within the upper surface of the body structure of the microfluidic system. Additionally, at least one member of the plurality of the library storage elements of the microfluidic system comprises a dried or immobilized test compound. Optionally, substantially all members of the plurality of library storage elements of the microfluidic system comprise a different dried or immobilized test compound. Alternatively, the library storage elements of the microfluidic system comprise dried or immobilized test compounds which are not all substantially different compounds. Furthermore, the plurality of library storage elements of the microfluidic system can optionally comprise a library of test compounds. At least one member, or substantially all members, of the plurality of microscale channels of the microfluidic system optionally contains a fluidic material, which fluidic material can optionally comprise a buffer. The microfluidic system of the invention can also have a fluid delivery system comprising a pipettor device. The fluid delivery system of the microfluidic system can optionally deliver volumes of about 20 microliters or less, of about 5 microliters or less, of about 1 microliter or less, of about 200 nanoliters or less, of about 50 nanoliters or less, of about 10 nanoliters or less, of about 2 nanoliters or less, or of about 1 nanoliter or less. The fluid delivered by the fluid delivery system can optionally comprise a buffer. In some aspects, the fluid delivery system simultaneously delivers a portion of fluid to about 2 to about 1,000,000 or more library storage elements, to about 100 to about 100,000 or more library storage elements, to about 1,000 to about 10,000 or more library storage elements, to about at least 2 to about 5 or more, to about at least 2 to about 10 or more, or to about at least 2 to about 15 or more library storage elements. In some aspects it delivers the portion of fluid to one or more library storage elements about every 1 minute or less, about every 30 seconds or less, about every 10 seconds or less, about every 5 seconds or less, or about every 1 second or less.

Additionally, the microfluidic system of the invention can further comprise a fluid direction system operably coupled to the plurality of microscale channels. Such fluid direction system can direct one or more of: movement of a first fluidic material through one or more member of the plurality of microscale channels; delivery of a second fluidic material to one or more member of the plurality of microscale reservoirs; movement of the second fluid material from the one or more member of the plurality of microscale reservoirs into one or more member of the plurality of microscale channels; movement of the second fluid material from the one or more member of the plurality of microscale reservoirs into one or more test-microchannel and thence into one or more member of the plurality of microscale channels; or movement of the first fluidic material through one or more test-microchannel.

In some aspects, the fluid direction system of the invention optionally directs the movement of a first fluidic material through a microscale channel of the microfluidic system to a microscale reservoir where the first fluidic material optionally contacts a test compound (optionally a dried or otherwise immobilized test compound) disposed within the microscale reservoir or wherein the first fluidic material does not contact the test compound within the microscale reservoir; delivery of a second fluidic material from the fluid delivery system to the microscale reservoir; and movement of the second fluidic material from the reservoir through the connected microscale channel.

In other aspects, the fluid direction system of the invention optionally directs the movement of a fluidic material through a microscale channel of the microfluidic system to a test-microchannel where the fluidic material contacts a test compound (optionally a dried or otherwise immobilized test compound) disposed within the test-microchannel; delivery of a second fluidic material from the fluid delivery system to the microscale reservoir; movement of the second fluidic material from the reservoir through the test-microchannel and through the connected microscale channel.

In yet other aspects, the fluid direction system of the invention optionally directs the movement of a fluidic material through a microscale channel of the microfluidic system to a test-microchannel where the fluidic material contacts a test compound (optionally a dried or otherwise immobilized test compound) disposed within the test-microchannel.

The present invention also includes a method of loading a plurality of test compounds from a plurality of microscale reservoirs into a microchannel system that is fluidly coupled to the plurality of microscale reservoirs. Such method of loading optionally comprises flowing a fluidic material through a microchannel to a microscale reservoir that contains a test-compound disposed within the microscale reservoir, delivering a second fluidic material to the microscale reservoir and flowing the second fluidic material from the microscale reservoir through a microchannel into the microchannel system, thereby loading the test-compound into the microchannel system. Such steps of loading a plurality of test compounds are optionally repeated and are optionally repeated for substantially all members of the plurality of test compounds. Additionally, the delivery of the second fluidic material to the microscale reservoir optionally is done by hand pipetting or robotic pipetting.

In other aspects, the invention includes a method of loading a plurality of test compounds from a plurality of test-microchannels into a microchannel system that is fluidly coupled to the plurality of test-microchannels. Such method of loading optionally comprises flowing a fluidic material through a microchannel to a test-microchannel that contains a test-compound disposed within the test-microchannel, delivering a second fluidic material to a microscale reservoir that is fluidly connected with the test-microchannel and flowing the second fluidic material from the microscale reservoir through the test-microchannel and the microscale channel into the microchannel system, thereby loading the test-compound into the microchannel system. Such steps of loading a plurality of test compounds are optionally repeated and are optionally repeated for substantially all members of the plurality of test compounds. Additionally, the delivery of the second fluidic material to the microscale reservoir optionally is done by hand pipetting or by robotic pipetting.

In yet other aspects, the invention includes a method of loading a plurality of test compounds from a plurality of test-microchannels into a microchannel system that is fluidly coupled to the plurality of test-microchannels. Such method of loading optionally comprises flowing a fluidic material through a microchannel to a test-microchannel that contains a test compound disposed therein, and flowing the fluidic material from the test-microchannel through a microscale channel into the microchannel system, thereby loading the test compound into the microchannel system. Such steps of loading a plurality of test compounds are optionally repeated and are optionally repeated for substantially all members of the plurality of test compounds.

Additionally, the various aspects of methods of loading of a plurality of test compounds from a plurality of microscale reservoirs or test-microchannels optionally comprise loading between about 1 to about 1,000,000 test compounds into the microchannel system, between about 10 to about 100,000 test compounds into the microchannel system, between about 100 to about 10,000 test compounds into the microchannel system, or between about 1,000 to about 5,000 test compounds. Furthermore, the various aspects of methods of the invention of loading of a test compound optionally comprise loading the test compound into the microchannel system from between about 2 to about 1,000,000 microscale reservoirs or test-microchannels, between about 10 to about 100,000 microscale reservoirs or test-microchannels, between about 100 to about 10,000 microscale reservoirs or test-microchannels, or between about 1,000 to about 5,000 microscale reservoirs or test-microchannels.

In another aspect, the various aspects of methods of loading a plurality of test compounds comprise wherein the microchannel system comprises a plurality of microscale channels disposed within a microfluidic device wherein one or more member of the plurality of microscale channels is fluidly coupled to one or more member of the plurality of microscale reservoirs or test-microchannels. Additionally and optionally the loading of a plurality of test compounds comprises substantially filling substantially all members of the plurality of microchannels with the first fluidic material.

In a further aspect of the invention, loading of test compounds comprises introducing a first fluidic material into the microchannel system and allowing the first fluidic material to flow through substantially all microchannels disposed within the microchannel system.

In the loading of test compounds from a plurality of microscale reservoirs or test-microchannels, flowing the first fluidic material optionally comprises electrokinetically flowing, flowing by use of pressure or flow through use of capillary or wicking forces.

Optionally, in the loading of test compounds from a plurality of microscale reservoirs or test-microchannels, either the first fluidic material and/or the second fluidic material comprises a buffer material. Optionally, such first fluidic material dissolves the first test compound, or, optionally, such second fluidic material dissolves the first test compound.

In some aspects of the invention, the method of loading a plurality of test compounds from a plurality of microscale reservoirs or test-microchannels involves delivering to the first microscale reservoir from about less than 20 microliters of the first or second fluidic material, less than about 5 microliters, less than about 1 microliter, less than about 200 nanoliters, less than about 50 nanoliters, less than about 10 nanoliters, less than about 2 nanoliters, or about 1 nanoliter or less. Additionally, the flowing of the second fluidic material comprises flowing via electrokinetic forces, flowing under pressure, or flowing using capillary or wicking forces and the second fluidic material is delivered to a microscale reservoir optionally about every 1 minute or less, about every 30 seconds or less, about every 10 seconds or less, about every 5 seconds or less, or about every 1 second or less. Furthermore, the second fluidic material is optionally delivered concurrently to between at least 2 members and 1,000,000, between at least 100 and 100,000 members, or between at least 1,000 and 10,000 members or more of the plurality of microscale reservoirs.

In yet another aspect of the invention of loading a plurality of test compounds from a plurality of microscale reservoirs or test-microchannels, the first fluidic material and the second fluidic material optionally comprise the same material, and optionally each fluidic material comprises a buffer material.

Many additional aspects of the invention will be apparent upon review, including uses of the devices and systems of the invention, methods of manufacture of the devices and systems of the invention, kits for practicing the methods of the invention and the like. For example, kits comprising any of the devices or systems set forth above, or elements thereof, in conjunction with packaging materials (e.g., containers, sealable plastic bags etc.) and instructions for using the devices, e.g., to practice the methods herein, are also contemplated.

### BRIEF DESCRIPTION OF THE FIGURES

FIG. 1, panels A, and B are schematic side views of optional library storage elements of the invention.

FIG. 2, panels A and B are schematic views of optional microchannel configurations of the invention.

FIG. 3, is a schematic representation of an optional heating arrangement involving an optional microchannel configuration of the invention.

FIG. 4, is a schematic diagram of an optional library array arrangement and microfluidic system of the invention.

FIG. 5, panels A, B, and C are a schematic top view and side views of an example microfluidic system comprising the elements of the invention.

FIG. 6, is a schematic of a system comprising a computer, detector and temperature controller.

### DETAILED DISCUSSION OF THE INVENTION

The methods and devices of the invention directly address and solve problems associated with screening large reagent or combinatorial chemical libraries. Specifically, the invention provides devices and methods for arrangement and presentation of large numbers of molecules/compounds (e.g., potential pharmaceutical compounds) in a stable format for use in high throughput screening. The invention also provides systems involving and utilizing these devices and methods that allow control of, e.g., material flow, data gathering and analysis, various experiment parameters, etc. In short, using a microfluidic library device of the invention allows researchers to screen compounds and molecules more quickly while using less volume of reagents and storing the compounds and molecules in a stable storage array.

In the current invention, numerous test molecules can be stored and screened, e.g., for their possible interaction(s) with a specific target molecule. Such interaction(s) includes not only, e.g., receptor-ligand interactions, but also such things as nucleic acid-nucleic acid hybridization interactions, and can include both specific and nonspecific interaction. The methods and devices herein are flexible and allow the storage and screening of many different types of compounds and molecules. For example, both the target molecule(s) to be assayed and the test molecules to be screened against the target molecule can be any one or more of numerous molecules including, but not limited to, proteins (whether enzymatic or not), enzymes, nucleic acids (e.g., single-stranded, double-stranded, or triple-stranded), ligands, peptide nucleic acids, cofactors, receptors, substrates, antibodies, antigens, polypeptides, monomeric and multimeric proteins (either homomeric or heteromeric), co-enzymes, co-factors, lipids, phosphate groups, oligosaccharides, prosthetic groups, synthetic oligonucleotides, portions of recombinant DNA molecules or chromosomal DNA, or portions/pieces of proteins/peptides/receptors/etc.

Briefly, the methods and devices of the current invention involving reagent library arrays allow for storage of, and screening of, the interaction between large numbers or various molecules while minimizing reagent usage, maximizing throughput speed and allowing for ease of molecule/compound/reagent storage. Other microfluidic devices for use in high throughput screening have been detailed in, e.g., U.S. Pat. No. 5,942,443 issued Aug. 24, 1999, entitled “High Throughput Screening Assay Systems in Microscale Fluidic Devices” to J. Wallace Parce et al. (which is incorporated herein by reference for all purposes). Additionally, other various devices or systems have previously been used to bring samples of reagent libraries into such screening devices or systems (whether involving a microfluidic device or not). For example, a pipettor device or a similar element can introduce samples to a screening device or system after drawing the samples from a reagent library. Additionally, other screening systems have used such methods as pipetting library samples by hand or drawing samples from multiwell plates. The current invention differs from the above methods and devices in numerous ways. For example, the samples to be assayed in the current invention are contained within libraries within the microfluidic devices of the invention.

Other library screening systems have contained samples (e.g., reagents, compounds molecules and the like) to be screened in various arrangements and formats, e.g., in multiwell plates comprising fluid samples. The present invention, however, utilizes deposited samples in specific library storage elements such as micro-reservoirs and test-microchannels present within the microfluidic device itself. The deposited samples are optionally dried, but can also be immobilized in, e.g., matrices, or in other liquid formats, etc. The samples are optionally reconstituted (i.e., from their dried or otherwise stored or immobilized forms), selectively introduced into a microchannel network of the microfluidic device and screened against other compound(s) (optionally from an additional library(ies) of the microfluidic device) to test for and/or quantify possible interactions, etc.

The present invention also optionally includes various elements involved in, e.g., transporting the samples and reagents involved, reconstitution of dried or immobilized samples, temperature control, fluid transport mechanisms, detection and quantification of molecular interactions (e.g., fluorescence detectors), robotic devices for, e.g., positioning of components or devices involved, etc.

Methods and Devices of the Invention

Screening of molecules, compounds, etc. in microfluidic devices usually is done within one or more microchannels (sometimes referred to herein as microfluidic channels) or microreservoirs, etc. The term “microfluidic”, as used herein, refers to a device component, e.g., chamber, channel, reservoir, or the like, that includes at least one cross-sectional dimension, such as depth, width, length, diameter, etc. of from about 0.1 micrometer to about 500 micrometer. Examples of microfluidic devices are detailed in, e.g., U.S. Pat. No. 5,942,443 issued Aug. 24, 1999, entitled “High Throughput Screening Assay Systems in Microscale Fluidic Devices” to J. Wallace Parce et al. and U.S. Pat. No. 5,880,071 issued Mar. 9, 1999, entitled “Electropipettor and Compensation Means for Electrophoretic Bias” to J. Wallace Parce et al., both of which are incorporated herein by reference for all purposes. In general, microfluidic devices are planar in structure and are constructed from an aggregation of planar substrate layers wherein the fluidic elements, such as microchannels, etc., are defined by the interface of the various substrate layers. The microchannels, etc. are usually etched, embossed, molded, ablated or otherwise fabricated into a surface of a first substrate layer as grooves, depressions, or the like. A second substrate layer is subsequently overlaid on the first substrate layer and bonded to it in order to cover the grooves, etc. in the first layer, thus creating sealed fluidic components within the interior portion of the device. Additionally, open-well micro-reservoirs can be formed by making perforations in one or more substrate layer (preferably the second substrate layer) which perforation optionally can correspond to depressed micro-reservoir areas on the complementary layer (preferably the first substrate layer).

The layers of the microfluidic devices can be composed of numerous types of materials depending on the specific compounds, reagents, etc. to be assayed and, e.g., the various procedures involved such as transport etc. For example, the substrate layers can be composed of, e.g., silica-based materials (such as glass, quartz, silicon, fused silica, or the like), polymeric materials (such as polymethylmethacrylate, polycarbonate, polytetrafluoroethylene, polyvinylchloride, polydimethylsiloxane, polysulfone, polystyrene, polymethylpentene, polypropylene, polyethylene, polyvinylidine fluoride, acrylonitrile-butadiene-styrene copolymer, parylene or the like), ceramic materials, etc. Also, depending on the specific reaction parameters of the desired screenings and the specific reagents, samples, etc. involved, specific micro-reservoir areas or other areas can be lined with different substances than that of which the rest of the microfluidic device is composed.

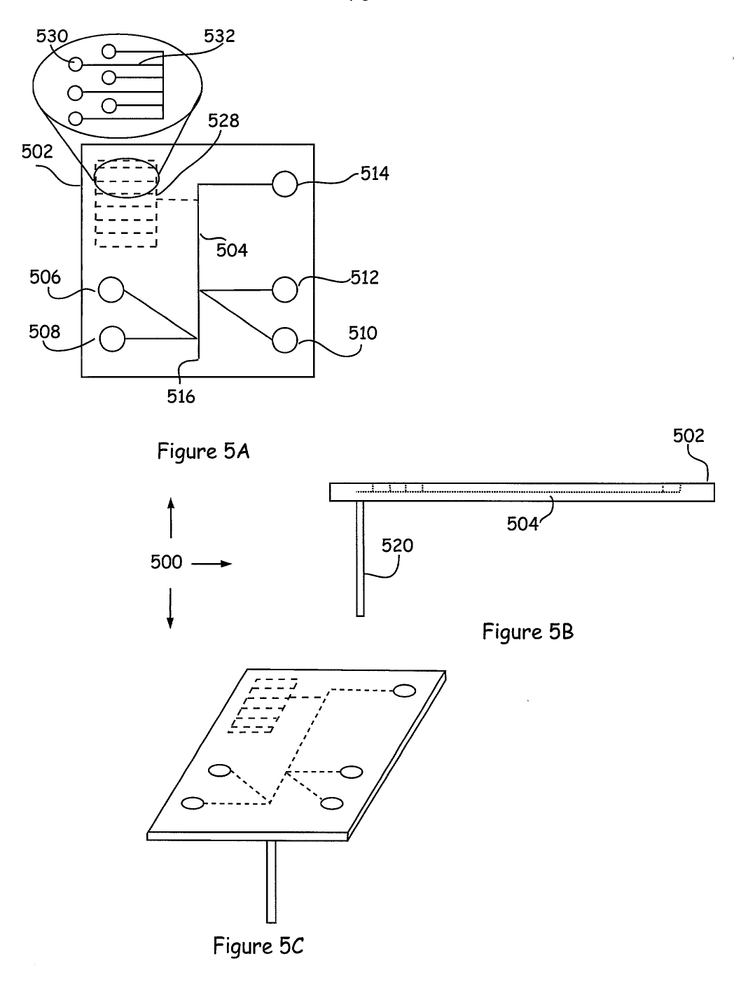

Although described in terms of a layered planar body structure, it will be appreciated that microfluidic devices in general and the present invention in particular can take a variety of forms, including aggregations of various fluidic components such as capillary tubes, individual chambers, arrangement of library array(s) etc., that are pieced together to provide the integrated elements of the complete device. For example, FIG. 5, illustrates one of many possible arrangements of the elements of the present invention. In one such possible arrangement, as shown in FIG. 5, body structure 502 has main channel 504 disposed therein, which is fluidly connected to various reservoirs that can optionally contain, e.g., buffer, reagents, etc. A library array containing individual library storage elements, is also fluidly connected to main channel 504. FIG. 5 is described, infra, in more detail. The microfluidic devices of the invention typically include at least one main analysis channel, but may include two or more main analysis channels in order to multiplex the number of analyses being carried out in the microfluidic device at any given time. Typically, a single device will include from about 1 to about 100 or more separate analysis channels or regions (e.g., 1,000 or more, 10,000 or more, etc.). Inmost cases, the analysis channel is intersected by at least one other microscale channel disposed within the body of the device. Typically, the one or more additional channels are used, e.g., to bring the samples, test compounds, assay reagents, etc. (any of which can optionally come from one or more library of the microfluidic device) into the main analysis channel, in order to carry out the desired assay.

Preparation of Reagent Library

Placement of Samples

In some aspects of the invention the samples (also referred to herein as “library samples”, “constituents”, or “library constituents”) that make up the library are provided dried upon or within the microfluidic device. Typically, such constituent samples are readily prepared by one or more of a variety of methods. For example, pipetting methods (e.g., by hand or by robot) are optionally used to place or “spot” the library constituents in discrete areas (i.e., library storage elements) of the microfluidic device (e.g., in the open-well micro-reservoirs). Alternatively, ink-jet printing methods or related methods are readily employable to print or place fluidic sample materials onto or within the library storage elements of the microfluidic device (again, e.g., in the open-well micro-reservoirs). See, e.g., U.S. Pat. No. 5,474,796 issued Dec. 12, 1995, entitled “Method and Apparatus for Conducting an Array of Chemical Reactions on a Support Surface” to Brennan. A broad range of printing methods suitable for use depositing samples within the libraries of current invention are known and can be readily adapted to use in the present invention (see also, e.g., U.S. Pat. No. 6,074,725, issued Jun. 13, 2000, entitled “Fabrication of Microfluidic circuits by Printing Techniques” to Kennedy for a discussion of printing materials, of, e.g., microscale elements, in the context of a microscale system). Additionally, samples can also be loaded into library arrays by pin or quill transfer (e.g., a pin or quill is dipped into a sample then contacted with the substrate surface thus transferring sample onto the library array). Any fluidic samples placed on or within the microfluidic device can optionally be lyophilized in place (see, e.g., U.S. Pat. No. 6,150,180, issued Nov. 21, 2000, entitled “High Throughput Screening Assay Systems in Microscale Fluidic Devices” to Parce, et al.). In other aspects, samples are dried on the library arrays by, e.g., freeze drying. This method produces dried samples that are generally in a more readily soluble from because of greater surface area. Additionally, depending upon the specific nature of the samples involved, other drying methods are used (e.g., heat, vacuum, use of a controlled atmosphere such as alkane or alcohol vapor, etc.). In some aspects, the library constituents optionally can be placed on or within the microfluidic device either before the substrate layers that comprise the microfluidic device, are joined or, alternatively, the library constituents can be placed on or within the microfluidic device after the substrate layers are joined together.

Alternatively, or additionally, the samples comprising the library samples can be immobilized in the library storage elements in the library array by methods other than drying. For example, porous matrices optionally can be used to retain fluid samples within discrete library storage elements of the device, e.g., micro-reservoirs and/or test-microchannels of the invention. Such sample materials are then removable by withdrawing the fluids from the pores of the substrate. Alternatively, sample materials may be coupled to matrices through numerous ways including, but not limited to, ionic, hydrophobic or hydrophilic interactions, severably covalent interactions (e.g., interactions that are severed through exposing the substrate to such things as high or low salt concentration, organic buffer, etc.) thermal dissociation or release (done by, e.g., using a matrix that incorporates a thermally responsive hydrogel, which expands or contracts upon heating thus expelling entrained library constituents), light or other electromagnetic radiation (used with, e.g., photolabile linker groups), etc. Furthermore, different library samples in the same library and/or in a different library on the same microfluidic chip can be deposited/immobilized in different fashions (e.g., any of the fashions described herein).

In order to aid in, e.g., sample deposition, drying, release or reconstitution, an excipient is optionally added to one or more library sample. Some non-limiting examples of useful excipients include, e.g., simple sugars (such as sucrose, fructose, maltose, trehelose, etc. as well as modified versions of such simple sugars), starches, dextrans, glycols (e.g., PEG and other polymers such as polyethylene oxide, polyvinylpyrrolidone, etc.), detergents, etc.

Multiple Withdrawals from Library Samples

In some aspects of the invention, the various library constituents in the library storage elements are present in sufficient quantities or, in some aspects of the invention, over a sufficiently large enough area, so as to permit multiple samplings of one or more of the different constituents. In some aspects, one or more library sample consists of an amount of material sufficient to allow withdrawal of that sample more than one time, preferably 2 or more times, three or more times, 5 or more times, or ten or more times. In general, each library sample is reconstituted with an amount of fluid (e.g., from an amount pipetted into a micro-reservoir or from an amount flowed into a micro-reservoir and/or test-microchannel, etc.) comprising 20 microliters or less, 5 microliters or less, 1 microliter or less, 200 nanoliters or less, 50 nanoliters or less, 25 nanoliters or less, 10 nanoliters or less, 2 nanoliters or less, or even 1 nanoliter or less.

Each amount of fluid deposited on (or contacted with) a library sample can optionally reconstitute only a portion of the sample. In other words, a portion of a specific library sample (as opposed to the entire specific library sample) can be reconstituted at any given time. Such partial reconstitution includes instances where the reconstituting fluid is only deposited upon (or is contacted with) a portion of the library sample, thus dissolving all of the library sample in that portion it contacts. Alternatively, the reconstituting fluid is deposited upon the entire specific library sample but the specific library sample is not completely reconstituted. In yet another alternative, a specific library constituent may be completely reconstituted, but only a portion of the reconstituted sample is flowed out of library storage element at a time.

For library constituents comprising selectively releasable compound materials (see, supra), a limited quantity of the library sample can be released by the controlled exposure of the material to the appropriate cleaving agent or environmental condition (e.g., light, heat, etc.) thus allowing multiple aliquots to be taken from a particular library sample. As a non-limiting example, if a library sample is linked to a storage matrix through a photocleavable linker, portions of the sample can be released by exposing the sample to varying degrees of photoexposure (i.e., adjusting the intensity and/or duration of photoexposure).

Composition of Samples

Typically, screening assays are performed on compounds that are present at concentrations in the micromolar range, e.g., from about 1 to about 20 micromolar. In the present invention, the library constituents are typically screened in volumes of the nanoliter range. Of course, depending upon the activity or efficacy of a given sample in, e.g., a particular screening system or other activity, this amount can vary greatly. Similarly, the amount of a given library sample can change significantly depending on the number of times the particular library sample is accessed. In general, each discrete quantity of library sample material will contain from between about 0.5 picograms or less to about 100 nanograms or more of sample material, between about 1 picogram or less to about 10 nanograms or more, between about 5 picograms or less to about 50 picograms or more, or between about 10 picograms or less to about 25 picograms or more. Alternatively, each discrete quantity of library sample material will contain from between about 1 femtomole or less to about 20 picomoles or more of sample material, between about 10 femtomoles or less to about 100 femtomoles or more, or between about 25 femtomoles or less to about 50 femtomoles or more. Typically, materials that are present in these amounts are more than adequate for at least 1 or more, at least 2 or more, at least 3 or more, at least 5 or more, or at least 10 or more aliquots from each library sample. The specific concentration and amount of each compound deposited upon the substrate surface typically depends upon the amount of material that is to be sampled, (which in turn depends upon the number of withdrawals to be taken from each library sample and the amount of sample to be taken in each withdrawal). Deposited compounds optionally can be present at quantities that are greater than or equal to about 1 picomole per square millimeter.

In order to facilitate rapid reconstitution of a library sample, in certain aspects it is preferred to provide the library sample in a thin layer on the surface of the substrate, or on the pores of the substrate (e.g., in a library storage element). For example, materials are typically deposited upon the substrate layers of the device at concentrations and quantities calculated substantially to provide a molecular monolayer or a near molecular monolayer of the compound species. In some cases, materials are deposited at greater than monolayer quantities, often falling between about one and twenty times monolayer quantities.

For a porous substrate, e.g., a honeycomb matrix, because the sample material is entrained in the porous matrix, the amount of surface area covered by a particular sample material is much greater per unit of external surface area than in the case of non-porous substrates. As such, much greater amounts of sample material can be provided in the same (or smaller) external surface area than in non-porous substrates.

Composition of Substrates Layers

As stated above, the substrates used to construct the microfluidic devices of the invention are typically fabricated from any number of different materials, depending upon, e.g., the nature of the library sample to be deposited thereon, the desired quantity of library samples to be deposited thereon, the specific reactions and/or interactions being assayed for, etc. For example, for some applications, the substrate can optionally comprise a solid non-porous material where the library sample is spotted or deposited upon the surface. Such substrates are typically suitable where it is less important to maximize the amount of library sample deposited on the substrate. Examples of such non-porous substrates include, e.g., metal materials, glass, quartz or silicon materials, polymer materials (or a polymer coating on a materials) including, e.g., polystyrene, polypropylene, polyethylene, polytetrafluoroethylene, polyearbonate, acrylics (e.g., polymethylmethacrylate), and the like.

The surface of a substrate layer may be of the same material as the non-surface areas of the substrate or, alternatively, the surface may comprise a coating on the substrate base. Furthermore, if the surface is coated, the coating optionally can cover either the entire substrate base or can cover select subparts of the substrate base, e.g., the surface of one or more library storage element. For example, in the case of glass substrates, the surface of the glass of the base substrate may be treated to provide surface properties that are compatible and/or beneficial to one or more library sample or reagent deposited thereon. Such treatments include derivatization of the glass surface, e.g., through silanization or the like, or through coating of the surface using, e.g., a thin layer of other material such as a polymeric or metallic material. Derivatization using silane chemistry is well known to those of skill in the art and can be readily employed to add, e.g., amine, aldehyde, or other functional groups to the surface of the glass substrate, depending upon the desired surface properties. Additionally, other non-glass substrates can comprise derivatized surfaces as well. Alternatively, a glass layer may be provided as a coating over the surface of another base substrate, e.g., silicon, metal, ceramic, or the like.

In the case of polymer substrates, as with the glass or other silica based substrates described herein, the substrate may be entirely comprised of the polymer materials, or the polymer materials may be provided as a coating over a support element (i.e., base substrate). Such base substrates include, but are not limited to metal, silicon, ceramic, glass, or other polymer or plastic and are used, e.g., to provide sufficient rigidity to the substrate. In some cases, metal substrates are optionally used, either coated or uncoated, in order to take advantage of their conductivity.

Further, in the case of metal substrates, metals that are not easily corroded under potentially high salt conditions, applied electric fields, and the like are optionally preferred. For this reason, titanium substrates, platinum substrates and gold substrates, for example, generally can be suitable, although other metals, e.g., aluminum, stainless steel, and the like, also can be useful. For cost reasons, titanium metal substrates are beneficial where no external coating is to be applied.

Alternatively, where greater amounts of material are desired to be immobilized upon a substrate, porous materials optionally can be used. Porous materials can provide an increased surface area upon which library samples can be immobilized, dried or otherwise disposed. Porous substrates include membranes, scintered materials, (e.g., metal, glass, polymers, etc.), spun polymer materials, or the like.

Examples of particularly useful porous substrate materials include substrate matrices such as aluminum oxide, etched polycarbonate substrates, etched silicon (optionally including a polymer or other suitable coating) and like substrates that comprise arrayed honeycomb pores, e.g., hexagonal pores. Such substrate matrices are used for their ability to maintain liquid samples within a confined area. Specifically, because of the matrix's porous nature, fluids deposited upon a surface of such a matrix do not laterally diffuse across the substrate surface to any great extent. Instead, the fluids wick into the pores in the substrate matrix. This property allows the library sample materials to be deposited upon the substrate matrix in relatively high densities without concern for diffusing of samples (e.g., out of a library storage element such as a micro-reservoir, test-microchannel, etc.). In addition, the pores in the substrate matrix provide a greatly increased surface area as compared to non-porous substrates, thus greater quantities of library sample material can be deposited than would otherwise be possible in a monolayer or similar thin coating.

Other useful materials for substrates include conventional porous membrane materials, e.g., nitrocellulose, polyvinylidine difluoride (PVDF), polysulfone, polyvinyl chloride, spun polypropylene, polytetrafluoroethylene (PTFE), and the like. However, honeycombed matrices are optionally more preferred as far as porous matrices are concerned, due to their ability to contain the deposited library samples within discrete sets of pores, rather than permitting their diffusion across or through the substrate matrix. Again, as with all of the substrate coatings discussed above, optionally either the entire substrate layer can be coated or only select regions (e.g., library storage elements) of the substrate base can be coated.

Configuration of Libraries

Library Storage Elements

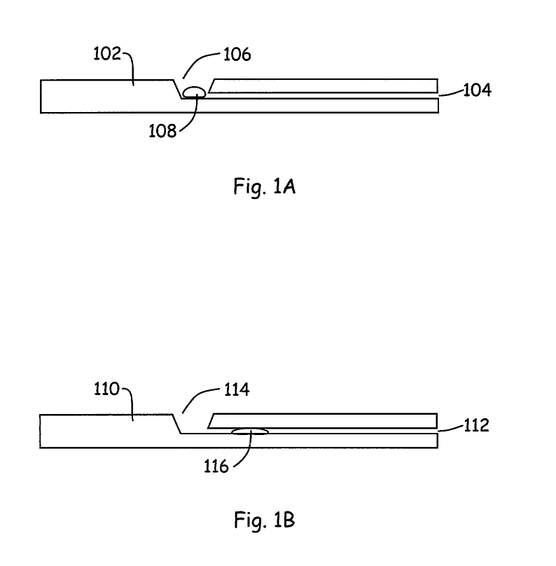

The samples which make up the libraries in the present invention can be deposited in numerous configurations within the microfluidic device. One preferred way of depositing library samples on or within the microfluidic device is by placing a sample in an open-well micro-reservoir as illustrated in FIG. 1A (also referred to herein as, e.g., microscale reservoirs, etc.). As shown, in cross-view, open-well micro-reservoir 106 is situated within substrate 102 of a microfluidic device. Micro-channel 104 connects open-well micro-reservoir 106 to the rest of the microfluidic device. Library sample 108 is shown within open-well micro-reservoir 106. The library sample is optionally deposited in a number of alternative embodiments such as dried, held within a matrix, etc. The shape of sample 108 as shown in FIG. 1A is for illustrative purposes only. Library samples can be present in numerous forms, such as in thin layers on the bottom and/or sides of an open-well micro-reservoir (e.g., reservoir 106).

Alternatively, the library samples can be deposited within a microchannel (i.e., a test-microchannel) which leads to an open-well micro-reservoir as is illustrated in cross-view in FIG. 1B. As illustrated, test-microchannel 112 is disposed within substrate 110 of a microfluidic device and connects open-well micro-reservoir 114 to the other areas of the microfluidic device (such as reaction channels, detection points, etc.). The library sample, 116, is disposed within the test-microchannel. A deposited sample in a test-microchannel (such as 116 in FIG. 1B) can optionally be in the form of a solid plug (e.g., of dried-down sample or sample immobilized within a matrix) or it can be in a form attached to the walls of the test-microchannel that leaves an opening (e.g., a lumen) through the deposited sample.

As can be seen from the above non-limiting illustrations, library samples in the current invention can be deposited in numerous manners and/or locations in library storage elements within the current microfluidic devices depending upon the specific needs of, e.g., the reagents/samples and experimental parameters being used.

Configuration of Library Arrays

The library samples in various aspects of the invention can be arrayed or arranged in numerous ways depending upon the individual requirements of the samples, reagents, assays, etc. involved in the desired screenings.

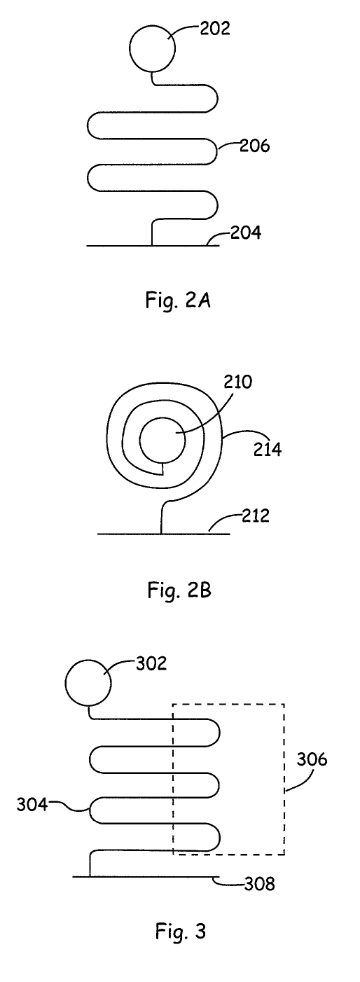

For example, a microchannel that connects a library storage element, e.g., a micro-reservoir to, e.g., a main analysis channel can be of varied design. Such a microchannel can be of different lengths, pathway shapes, etc. depending upon the appropriate screening parameters. Different microchannel pathway designs can be used for, e.g., preventing and/or decreasing unwanted contamination into the microchannel (e.g., from the main analysis channel) by acting as a diffusion barrier, or allowing long flow times between library storage elements in the sample library and, e.g., a main analysis channel. Such increased flow times can be used for library samples that are slow to reconstitute or which need longer time to, e.g., interact with another molecule or compound in the reconstituting buffer/solution. For example, FIG. 2 illustrates two non-limiting examples of such possible pathway designs. As shown in FIG. 2A, library storage element 202 is connected to a main analysis channel, 204, by microchannel 206. As stated above, the pathway of the microchannel 206 can be, e.g., convoluted in order to, e.g., increase the transit time between library storage element 202 and main analysis channel 204. Such an increase in transit time can be useful to, e.g., allow proper time for full reconstitution of a dried library sample, e.g., where the sample is in particulate form. FIG. 2B, illustrates another non-limiting example of a possible configuration of a microchannel leading from a library storage site. As show, library storage element 210 is connected to main analysis channel 212 by microchannel 214. Of course, in the examples herein, library storage elements can be any of the types listed herein, such as test-microchannels, micro-reservoirs, etc. and while a particular example may mention one specific sample storage type, unless otherwise mentioned, any storage type can be used.

As can thus be seen, the individual pathways for microchannels leading from library storage elements can be configured to carefully control such parameters as transit time between the storage area and, e.g., a reaction area where the library sample is interacted with one or more other molecules or compounds. Depending upon the parameters involved, the actual pathway of the microchannels can be of any design or footprint. Additionally, different microchannels leading from library storage elements of different library samples can be configured in different fashions in order to allow for, e.g., specific timing in loading or to take advantage of different properties of each sample. Furthermore, the configuration of microchannels leading from library storage elements can optionally be designed to allow an optimal number of library storage elements to fit into a given space within a microfluidic device.

In some optional embodiments, a reconstituted library sample can be heated and/or cooled one or more times by being flowed from a library storage element through a microchannel that traverses one or more areas of different temperature. FIG. 3 illustrates one possible microchannel configuration allowing temperature cycling of library samples. Microchannel 304 connects library storage element 302 and main analysis channel 308. Microchannel 304 lies both within and without of heated region 306, thus causing the library sample to cycle in temperature as it flows through microchannel 304. Variations in temperature cycling can be used in optional embodiments of the invention to, e.g., PCR amplify DNA regions from a library of, e.g., patient DNA before screening the library (i.e., the amplified portions of the library).

In some aspects, the current invention contains multi-analysis libraries wherein individual library constituents are fed into multiple experimental procedures, screenings, etc. For example, each constituent of a DNA library (e.g., where each sample comprises DNA from a pool of patients, etc.) can optionally be screened against multiple probes (e.g., probes to test for the presence of such things as various genetic diseases and/or the presence of DNA from diseases such as, e.g., hepatitis).

In other aspects, the current invention optionally includes multiple libraries incorporated into the same microfluidic device. Such ability allows for complex experimental design contained within the same microfluidic device. For example, as in the above illustration, one or more constituent of a DNA library (e.g., comprising a DNA sample from a pool of patients) can optionally be screened against one or more constituent of a probe library (e.g., comprising DNA probes for numerous genetic diseases, etc.). As a further option, the one or more constituent of the DNA library and/or of the probe library optionally can be PCR amplified before it is interacted with the one or more constituent of the opposing library.

As mentioned previously, the configuration of library storage elements and/or of microchannels leading from library storage elements can be manipulated to produce a desired density of library samples (i.e., in library storage elements) in a microfluidic device of the invention. In general, the devices of the present invention typically include a relatively high density of library storage elements per unit area. However, the density of library storage elements per unit area can be optimized depending upon the parameters of the particular number and types of assay(s) to be performed. For example, some embodiments of the invention can comprise a large number of library storage elements arrayed within a large area of the microfluidic device thus producing a low sample density (i.e., low number of samples/cm²). Other embodiments of the invention can comprise a small number of library storage elements arrayed within a small area of the microfluidic device thus producing a high sample density. In some embodiments, the library arrays of the invention can optionally comprise a library storage element density (or sample density) of between from about 5 library storage elements per square centimeter up to about 10,000 library storage elements per square centimeter, from about 100 library storage elements per square centimeter up to about 5,000 library storage elements per square centimeter, from about 1,000 library storage elements per square centimeter up to about 2,500 library storage elements per square centimeter, from about 100 to about 500 library storage elements per square centimeter, or from about 400 to about 4,000 library storage elements per square centimeter. Additionally, not only can different libraries within the same microfluidic device optionally have different library storage element densities, but the density within a single library can also vary (i.e., different areas within a library can have a greater number of library storage elements per unit area than other areas in that same library).

Illustrative Example of Sample Library Screening

As stated previously, the libraries of the current invention can be composed of numerous molecule types thus allowing for diverse, e.g., screening assays. For example optional embodiments of the invention can include, but are not limited to, one or more libraries comprising: proteins (whether enzymatic or not), enzymes, nucleic acids (e.g., single-stranded, double-stranded, triple-stranded), ligands, lipids, peptide nucleic acids, co-factors, receptors, substrates, antibodies, antigens, polypeptides, monomeric and multimeric proteins (either homomeric or heteromeric), coenzymes, phosphate groups, oligosaccharides, prosthetic groups, synthetic oligonucleotides, portions or recombinant DNA molecules or chromosomal DNA, and portions or fragments of any of the above.

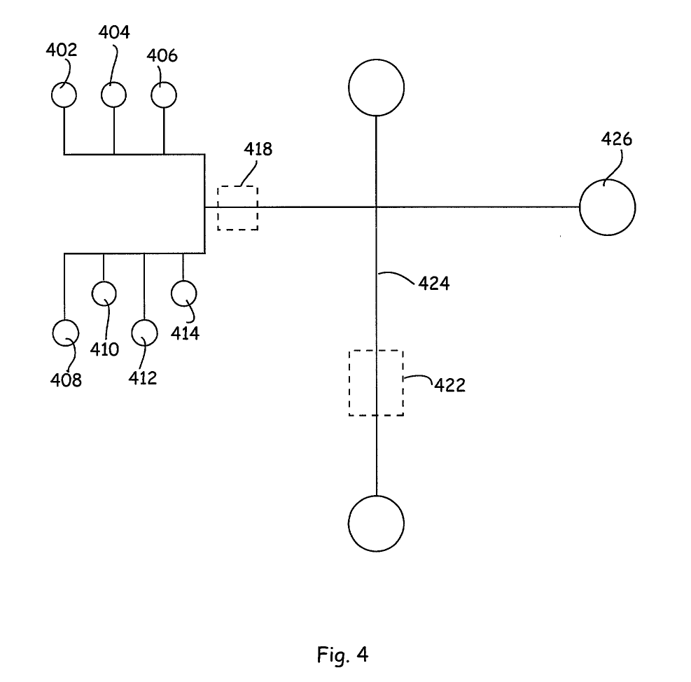

One non-limiting example of the current invention is shown in FIG. 4. The microfluidic device as shown in FIG. 4 comprises two sample libraries. The library represented by library storage sites 402, 404, and 406 optionally can comprise a variety of antibodies, while the library represented by library storage sites 408, 410, 412, and 414 optionally can comprise an array of putative antigens. As shown in FIG. 4, both the antibody library (containing 3 samples) and the antigen library (containing 4 samples) can optionally be increased in number of samples to include as many samples in each library as are necessary for the specific needs and parameters of the screening in question and which can be arranged within the space of the microfluidic device.

Through proper control of fluid flow within the microfluidic device each sample in the antibody library can be mixed with each sample in the antigen library and screened for recognition and binding. For example, the antibody deposited in library storage site 402 can be, e.g., reconstituted from its stored form (e.g., whether dried, liquid, or otherwise immobilized) and flowed into mixing region 418 where it can optionally mix with the putative antigen(s) from, e.g., library storage site 408 which itself has been reconstituted from its stored form. Proper reagents, etc. needed for detection of antibody-antigen interaction can optionally be added to the main analysis channel, 424, from, e.g., reagent wells 426, etc. thus allowing for detection of antibody-antigen interaction, if any, in detection area 422.

Integrated Systems, Methods and Microfluidic Devices of the Invention

The microfluidic devices of the invention include numerous optional variant embodiments including methods and devices for, e.g., fluid transport, temperature control, detection and the like. For example, a variety of microscale systems are optionally adapted for use with the devices and components comprising the libraries, etc. as discussed herein. These systems are described in numerous publications by the inventors and their coworkers. These include certain issued U.S. Patents, including U.S. Pat. Nos. 5,699,157 (J. Wallace Parce) issued Dec. 16, 1997, U.S. Pat. No. 5,779,868 (J. Wallace Parce et al.) issued Jul. 14, 1998, U.S. Pat. No. 5,800,690 (Calvin Y. H. Chow et al.) issued Sep. 1, 1998, U.S. Pat. No. 5,842,787 (Anne R. Kopf-Sill et al.) issued Dec. 1, 1998, U.S. Pat. No. 5,852,495 (J. Wallace Parce) issued Dec. 22, 1998, U.S. Pat. No. 5,869,004 (J. Wallace Parce et al.) issued Feb. 9, 1999, U.S. Pat. No. 5,876,675 (Colin B. Kennedy) issued Mar. 3, 2, 1999, 5,880,071 (J. Wallace Parce et al.) issued Mar. 9, 1999, U.S. Pat. No. 5,882,465 (Richard J. McReynolds) issued Mar. 16, 1999, U.S. Pat. No. 5,885,470 (J. Wallace Parce et al.) issued Mar. 23, 1999, U.S. Pat. No. 5,942,443 (J. Wallace Parce et al.) issued Aug. 24, 1999, U.S. Pat. No. 5,948,227 (Robert S. Dubrow) issued Sep. 7, 1999, U.S. Pat. No. 5,955,028 (Calvin Y. H. Chow) issued Sep. 21, 1999, U.S. Pat. No. 5,957,579 (Anne R. Kopf-Sill et al.) issued Sep. 28, 1999, U.S. Pat. No. 5,958,203 (J. Wallace Parce et al.) issued Sep. 28, 1999, U.S. Pat. No. 5,958,694 (Theo T. Nikiforov) issued Sep. 28, 1999, U.S. Pat. No. 5,959,291 (Morten J. Jensen) issued Sep. 28, 1999, U.S. Pat. No. 5,964,995 (Theo T. Nikiforov et al.) issued Oct. 12, 1999, 5,965,001 (Calvin Y. H. Chow et al.) issued Oct. 12, 1999, 5,965,410 (Calvin Y. H. Chow et al.) issued Oct. 12, 1999, 5,972,187 (J. Wallace Parce et al.) issued Oct. 26, 1999, U.S. Pat. No. 5,976,336 (Robert S. Dubrow et al.) issued Nov. 2, 1999, U.S. Pat. No. 5,989,402 (Calvin Y. H. Chow et al.) issued Nov. 23, 1999, U.S. Pat. No. 6,001,231 (Anne R. Kopf-Sill) issued Dec. 14, 1999, U.S. Pat. No. 6,011,252 (Morten J. Jensen) issued Jan. 4, 2000, 6,012,902 (J. Wallace Parce) issued Jan. 11, 2000, 6,042,709 (J. Wallace Parce et al.) issued Mar. 28, 2000, U.S. Pat. No. 6,042,710 (Robert S. Dubrow) issued Mar. 28, 2000, U.S. Pat. No. 6,046,056 (J. Wallace Parce et al.) issued Apr. 4, 2000, U.S. Pat. No. 6,048,498 (Colin B. Kennedy) issued Apr. 11, 2000, U.S. Pat. No. 6,068,752 (Robert S. Dubrow et al.) issued May 30, 2000, U.S. Pat. No. 6,071,478 (Calvin Y. H. Chow) issued Jun. 6, 2000, U.S. Pat. No. 6,074,725 (Colin B. Kennedy) issued Jun. 13, 2000, U.S. Pat. No. 6,080,295 (J. Wallace Parce et al.) issued Jun. 27, 2000, U.S. Pat. No. 6,086,740 (Colin B. Kennedy) issued Jul. 11, 2000, U.S. Pat. No. 6,086,825 (Steven A. Sundberg et al.) issued Jul. 11, 2000, U.S. Pat. No. 6,090,251 (Steven A. Sundberg et al.) issued Jul. 18, 2000, U.S. Pat. No. 6,100,541 (Robert Nagle et al.) issued Aug. 8, 2000, U.S. Pat. No. 6,107,044 (Theo T. Nikiforov) issued Aug. 22, 2000, U.S. Pat. No. 6,123,798 (Khushroo Gandhi et al.) issued Sep. 26, 2000, U.S. Pat. No. 6,129,826 (Theo T. Nikiforov et al.) issued Oct. 10, 2000, U.S. Pat. No. 6,132,685 (Joseph E. Kersco et al.) issued Oct. 17, 2000, U.S. Pat. No. 6,148,508 (Jeffrey A. Wolk) issued Nov. 21, 2000, U.S. Pat. No. 6,149,787 (Andrea W. Chow et al.) issued Nov. 21, 2000, U.S. Pat. No. 6,149,870 (J. Wallace Parce et al.) issued Nov. 21, 2000, U.S. Pat. No. 6,150,119 (Anne R. Kopf-Sill et al.) issued Nov. 21, 2000, U.S. Pat. No. 6,150,180 (J. Wallace Parce et al.) issued Nov. 21, 2000, U.S. Pat. No. 6,153,073 (Robert S. Dubrow et al.) issued Nov. 28, 2000, U.S. Pat. No. 6,156,181 (J. Wallace Parce et al.) issued Dec. 5, 2000, U.S. Pat. No. 6,167,910 (Calvin Y. H. Chow) issued Jan. 2, 2001, U.S. Pat. No. 6,171,067 (J. Wallace Parce) issued Jan. 9, 2001, U.S. Pat. No. 6,171,850 (Robert Nagle et al.) issued Jan. 9, 2001, U.S. Pat. No. 6,172,353 (Morten J. Jensen) issued Jan. 9, 2001, U.S. Pat. No. 6,174,675 (Calvin Y. H. Chow et al.) issued Jan. 16, 2001, U.S. Pat. No. 6,182,733 (Richard J. McReynolds) issued Feb. 6, 2001, U.S. Pat. No. 6,186,660 (Anne R. Kopf-Sill et al.) issued Feb. 13, 2001, U.S. Pat No. 6,221,226 (Anne R. Kopf-Sill) issued Apr. 24, 2001, and U.S. Pat. No. 6,233,048 (J. Wallace Parce) issued May 15, 2001.

Systems adapted for use with the devices and components comprising the libraries, etc. of the present invention are also described in, e.g., various published PCT applications, such as, WO 98/00231, WO 98/00705, WO 98/00707, WO 98/02728, WO 98/05424, WO 98/22811, WO 98/45481, WO 98/45929, WO 98/46438, and WO 98/49548, WO 98/55852, WO 98/56505, WO 98/56956, WO 99/00649, WO 99/10735, WO 99/12016, WO 99/16162, WO 99/19056, WO 99/19516, WO 99/29497, WO 99/31495, WO 99/34205, WO 99/43432, WO 99/44217, WO 99/56954, WO 99/64836, WO 99/64840, WO 99/64848, WO 99/67639, WO 00/07026, WO 00/09753, WO 00/10015, WO 00/21666, WO 00/22424, WO 00/26657, WO 00/42212, WO 00/43766, WO 00/45172, WO 00/46594, WO 00/50172, WO 00/50642, WO 00/58719, WO 00/60108, WO 00/70080, WO 00/70353, WO 00/72016, WO 00/73799, WO 00/78454, WO 01/02850, WO 01/14865, WO 01/17797, and WO 01/27253.

As used herein, the term “microfluidic device” refers to a system or device having fluidic conduits or chambers that are generally fabricated at the micron to sub-micron scale, e.g., typically having at least one cross-sectional dimension in the range of from about 0.1 micrometer to about 500 micrometer. The microfluidic system of the current invention is fabricated from materials that are compatible with the conditions present in the specific experiments, the specific library samples, reagents, etc. under examination, etc. Such conditions include, but are not limited to, pH, temperature, ionic concentration, pressure, and application of electrical fields. The materials of the device are also chosen for their inertness to components of the experiments to be carried out in the device. Such materials include, but are not limited to, glass, quartz, silicon, and polymeric substrates, e.g., plastics, depending on the intended application.

Although the devices and systems specifically illustrated herein are generally described in terms of the performance of a few or of one particular operation, it will be readily appreciated from this disclosure that the flexibility of these systems permits easy integration of additional operations and devices. For example, the devices and systems described will optionally include structures, reagents and systems for performing virtually any number of operations both upstream and downstream from the operations specifically described herein (e.g., storage, reconstitution, and use of the sample library constituents, etc.). Such upstream operations include such operations as sample handling and preparation, e.g., cell separation, extraction, purification, amplification, cellular activation, labeling reactions, dilution, aliquotting, and the like involving either library constituents and/or compounds, reagents, etc. that are not library constituents. Similarly, downstream operations optionally include similar operations, including, e.g., separation of sample components, labeling of components, assays and detection operations, electrokinetic or pressure-based injection of components or the like. Assay and detection operations include, without limitation, cell fluorescence assays, cell activity assays, probe interrogation assays, e.g., nucleic acid hybridization assays utilizing individual probes, free or tethered within the channels or chambers of the device and/or probe arrays having large numbers of different, discretely positioned probes, receptor/ligand assays, immunoassays, and the like. Any of these elements are optionally fixed to, e.g., channel walls, or the like. An example system is described below.

The microfluidic devices of the present invention can include other features of microscale systems, such as fluid transport systems. Such systems, e.g., direct particle/fluid movement within, and to, the microfluidic devices as well as directing the flow of fluids to reconstitute the library constituents at the library storage elements and flow of reconstituted library samples (as well as other fluidic components such as reagents, etc.). Such fluid transport systems can incorporate any movement mechanism set forth herein (e.g., fluid pressure sources for modulating fluid pressure in microchannels/micro-reservoirs/etc.; electrokinetic controllers for modulating voltage or current in the microchannels/micro-reservoirs/etc.; gravity flow modulators; magnetic control elements for modulating a magnetic field within the microfluidic device; use of hydrostatic, capillary, or wicking forces; or combinations thereof.

The microfluidic devices of the invention can also include fluid manipulation elements such as a parallel stream fluidic converter, i.e., a converter which facilitates conversion of at least one serial stream of reagents into parallel streams of reagents for parallel delivery to a reaction site or reaction sites within the device. The systems herein optionally include mechanisms such as a valve manifold and a plurality of solenoid valves to control flow switching, e.g., between channels and/or to control pressure/vacuum levels in the, e.g., microchannels (such as analysis or incubation channels or channels leading to library storage sites). Another example of a fluid manipulation element includes, e.g., a capillary optionally used to sip a non-library sample(s) or reagent, etc. from a microtiter plate and to deliver it to one of a plurality of channels, e.g., parallel reaction or assay channels. Additionally, molecules, etc. are optionally loaded into one or more channels of a microfluidic device through one or more capillary element fluidly coupled to each of one or more channels and to a sample or particle source, such as a microwell plate. However, the methods and devices of the invention typically and/or optionally function without the use of any outside storage access (e.g., of a microwell plate via a capillary element, etc.).

In the present invention, materials such as cells, proteins, antibodies, enzymes, substrates, buffers, or the like are optionally monitored and/or detected so that, e.g., the presence of a component of interest can be detected, an activity of a compound can be determined, or an effect of a modulator, e.g., on an enzyme's activity, can be measured. Depending upon the detected signal measurements, decisions are optionally made regarding subsequent fluidic operations, e.g., whether to assay a particular component in detail to determine, e.g., kinetic information or, e.g., whether a sample from a first library is to be assayed against one or more, or a specific, sample from another library.

In brief, the systems described herein optionally include microfluidic devices, as described above, in conjunction with additional instrumentation for controlling fluid transport, flow rate, and direction within the devices; detection instrumentation for detecting or sensing results of the operations performed by the system; processors, e.g., computers, for instructing the controlling instrumentation in accordance with preprogrammed instructions, receiving data from the detection instrumentation, and for analyzing, storing and interpreting the data, and for providing the data and interpretations in a readily accessible reporting format.

Temperature Control

The present invention can control temperatures to control reaction parameters, e.g., in thermocycling reactions (e.g., PCR, LCR), or to control reagent properties or to help in the reconstitution of library samples, etc. In general, and in optional embodiments of the invention, various heating methods can been used to provide a controlled temperature in miniaturized fluidic systems. Such heating methods include both joule and non-joule heating.

Non-joule heating methods can be internal, i.e., integrated into the structure of the microfluidic device, or external, i.e., separate from the microfluidic device. Non-joule heat sources can include, e.g., photon beams, fluid jets, liquid jets, lasers, electromagnetic fields, gas jets, electron beams, thermoelectric heaters, water baths, furnaces, resistive thin films, resistive heating coils, peltier heaters, or other materials, which provide heat to the fluidic system in a conductive manner. The conductive heating elements transfer thermal energy from, e.g., a resistive element in the heating element to the microfluidic system by way of conduction. Thermal energy provided to the microfluidic system overall, increases the temperature of the microfluidic system to a desired temperature. Accordingly, the fluid temperature and the temperature of the molecules within, e.g., the microchannels of the system, the library arrays of the system, etc. is also increased. An internal controller in the heating element or within the microfluidic device optionally can be used to regulate the temperature involved. These examples are not limiting and numerous other energy sources can be utilized to raise the fluid temperature in the microfluidic device.

Non-joule heating units can attach directly to an external portion of the microfluidic device. Alternatively, non-joule heating units can be integrated into the structure of the microfluidic device. In either case, the non-joule heating is optionally applied to only selected portions of the microfluidic devices (e.g., such as microchannels leading from library storage elements and/or reaction areas, detection areas, etc.) or optionally heats the entire microfluidic device and provides a uniform temperature distribution throughout the device.

A variety of methods can be used to lower fluid temperature in the microfluidic system, e.g., through use of energy sinks. Such an energy sink can be a thermal sink or a chemical sink and can be flood, time-varying, spatially varying, or continuous. The thermal sink can include, among others, a fluid jet, a liquid jet, a gas jet, a cryogenic fluid, a super-cooled liquid, a thermoelectric cooling means, e.g., peltier device or an electromagnetic field.

In general, electric current passing through the fluid in a channel produces heat by dissipating energy through the electrical resistance of the fluid. Power dissipates as the current passes through the fluid and goes into the fluid as energy as a function of time to heat the fluid. The following mathematical expression generally describes a relationship between power, electrical current, and fluid resistance: where POWER=power dissipated in fluid; I=electric current passing through fluid; and R=electric resistance of fluid.

POWER=I²R

The above equation provides a relationship between power dissipated (“POWER”) to current (“I”) and resistance (“R”). In some of the embodiments of the invention, wherein electric current is directed toward moving a fluid, a portion of the power goes into kinetic energy of moving the fluid through the channel. Joule heating uses a selected portion of the power to heat the fluid in the channel or selected channel region(s) of the microfluidic device and can utilize in-channel electrodes. See, e.g., U.S. Pat. No. 5,965,410, which is incorporated herein by reference in its entirety for all purposes. Such a channel region is often narrower or smaller in cross sectional area than other channel regions in the channel structure. The small cross sectional area provides higher resistance in the fluid, which increases the temperature of the fluid as electric current passes therethrough. Alternatively, the electric current can be increased along the length of the channel by increased voltage, which also increases the amount of power dissipated into the fluid to correspondingly increase fluid temperature.

Joule heating permits the precise regional control of temperature and/or heating within separate microfluidic elements of the device of the invention, e.g., within one or several separate channels, without heating other regions where such heating is, e.g., undesirable. Because the microfluidic elements are extremely small in comparison to the mass of the entire microfluidic device in which they are fabricated, such heat remains substantially localized, e.g., it dissipates into and from the device before it affects other fluidic elements. In other words, the relatively massive device functions as a heat sink for the separate fluidic elements contained therein.

To selectively control the temperature of fluid or material of a region of, e.g., a microchannel, the joule heating power supply of the invention can apply voltage and/or current in several optional ways. For instance, the power supply optionally applies direct current (i.e., DC), which passes through one region of a microchannel and into another region of the same microchannel which is smaller in cross sectional area in order to heat fluid and material in the second region. This direct current can be selectively adjusted in magnitude to complement any voltage or electric field applied between the regions to move materials in and out of the respective regions.

In order to heat the material within a region, without adversely affecting the movement of a material, alternating current (i.e., AC) can be selectively applied by the power supply. The AC used to heat the fluid can be selectively adjusted to complement any voltage or electric field applied between regions in order to move fluid in and out of various regions of the device. Alternating current, voltage, and/or frequency can be adjusted, for example, to heat a fluid without substantially moving the fluid.

Alternatively, the power supply can apply a pulse or impulse of current and/or voltage, which will pass through one microchannel region and into another microchannel region to heat the fluid in the region at a given instance in time. This pulse can be selectively adjusted to complement any voltage or electric field applied between the regions in order to move materials, e.g., fluids or other materials, into and out of the various regions (e.g., flowing reconstituted library samples through microchannels). Pulse width, shape, and/or intensity can be adjusted, for example, to heat the fluid substantially without moving the fluids or materials, or to heat the material while moving the fluid or materials. Still further, the power supply optionally applies any combination of DC, AC, and pulse, depending upon the application. The microchannel(s) itself optionally has a desired cross sectional area and/or profile (e.g., diameter, width or depth) that enhances the heating effects of the current passed through it and the thermal transfer of energy from the current to the fluid.

Because electrical energy is optionally used to control temperature directly within the fluids contained in the microfluidic devices, the invention is optionally utilized in microfluidic systems that employ electrokinetic material transport systems, as noted herein. Specifically, the same electrical controllers, power supplies and electrodes can be readily used to control temperature contemporaneously with their control of material transport. In some embodiments of the invention, the device provides multiple temperature zones by use of zone heating. On such example apparatus is described in Kopp, M. et al. (1998) “Chemical amplification: continuous-flow PCR on a chip” Science 280(5366):1046-1048. The apparatus described therein consists of a chip with three temperature zones, corresponding to denaturing, annealing, and primer extension temperatures for PCR. A channel fabricated into the chip passes through each zone multiple times to effect a 20 cycle PCR. By changing the flow rate of fluids through the chip, Kopp et al., were able to change the cycle time of the PCR. While devices used for the present invention can be similar to that described by Kopp, they typically differ in significant ways. For example, the reactions performed by Kopp were limited to 20 cycles, which was a fixed aspect of the chip used in their experiments. In the present invention, reactions optionally comprise any number of cycles (e.g., depending on the parameters of the specific molecules being assayed). For example library samples comprising DNA can be PCR amplified for any number of desired cycles.

As can be seen from the above, the current invention can be configured in many different arrangements depending upon the specific needs of the molecules under consideration (e.g., both the molecules that comprise the libraries and any additional molecules, e.g., that are to be interacted with the library samples). Again, the above non-limiting illustrations are only examples of the many different configurations/embodiments of the invention.

Fluid Flow

A variety of controlling instrumentation and methodology is optionally utilized in conjunction with the microfluidic devices described herein, for controlling the transport and direction of fluidic materials and/or materials within the devices of the present invention by, e.g., pressure-based or electrokinetic control, etc.

In the present system, the fluid direction system controls the transport, flow and/or movement of samples (e.g., reconstituted library components), other reagents (e.g., buffers to reconstitute library components), etc. into and through the microfluidic device. For example, the fluid direction system optionally directs the movement of one or more buffer, fluid, etc. into a library storage element, where the fluid optionally reconstitutes a stored library sample. The fluid direction system also optionally directs the simultaneous or sequential movement of one or more reconstituted library sample into a detection region and optionally to and from, e.g., reagent reservoirs, waste reservoirs, etc. Additionally, the fluid direction system can optionally direct the loading and unloading of reagents, samples not contained in libraries, and other fluids, etc. in the devices of the invention.

The fluid direction system also optionally iteratively repeats the fluid direction movements to create high throughput screening, e.g., of thousands of samples. Alternatively, the fluid direction system repeats the fluid direction movements to a lesser degree of iterations to create a low throughput screening (applied, e.g., when the specific analysis under observation requires a long incubation time when a high throughput format would be counter-productive) or the fluid direction system utilizes a format of high throughput and low throughput screening depending on the specific requirements of the assay. Additionally, the devices of the invention optionally use a multiplex format to achieve high throughput screening, e.g., through use of a series of multiplexed sipper devices (e.g., to take up multiple buffer types, etc.) or multiplexed system of channels coupled to a single controller for screening in order to increase the amount of samples analyzed in a given period of time. Furthermore, the devices of the invention optionally utilize multiple libraries on the same chip, thus allowing for multiple analyses to proceed simultaneously or for sequential or cascade analyses to occur. Again, the fluid direction system of the invention optionally controls the flow (timing, rate, etc.) of samples, reagents, buffers, etc. involved in the various optional multiplex embodiments of the invention.

One method of achieving transport or movement of particles through microfluidic channels is by electrokinetic material transport. In general, electrokinetic material transport and direction systems include those systems that rely upon the electrophoretic mobility of charged species within the electric field applied to the structure. Such systems are more particularly referred to as electrophoretic material transport systems.

Electrokinetic material transport systems, as used herein, and as optional aspects of the present invention, include systems that transport and direct materials within a structure containing, e.g., microchannels, micro-reservoirs, library storage elements, etc., through the application of electrical fields to the materials, thereby causing material movement through and among the areas of the microfluidic devices, e.g., cations will move toward a negative electrode, while anions will move toward a positive electrode. Movement of fluids toward or away from a cathode or anode can cause movement of particles suspended within the fluid (or even particles over which the fluid flows). Similarly, the particles can be charged, in which case they will move toward an oppositely charged electrode (indeed, it is possible to achieve fluid flow in one direction while achieving particle flow in the opposite direction). In some embodiments of the present invention, the fluid and/or particles, etc. within the fluid, can be immobile or flowing.

For optional electrokinetic applications of the present invention, the walls of interior channels of the electrokinetic transport system are optionally charged or uncharged. Typical electrokinetic transport systems are made of glass, charged polymers, and uncharged polymers. The interior channels are optionally coated with a material which alters the surface charge of the channel. A variety of electrokinetic controllers are described, e.g., in Ramsey WO 96/04547, Parce et al. WO 98/46438 and Dubrow et al., WO 98/49548 (all of which are incorporated herein by reference in their entirety for all purposes), as well as in a variety of other references noted herein.

To provide appropriate electric fields, the system of the microfluidic device optionally includes a voltage controller that is capable of applying selectable voltage levels, simultaneously, to, e.g., each of the various microchannels and micro-reservoirs. Such a voltage controller is optionally implemented using multiple voltage dividers and multiple relays to obtain the selectable voltage levels. Alternatively, multiple independent voltage sources are used. The voltage controller is optionally electrically connected to each of the device's fluid conduits via an electrode positioned or fabricated within each of the plurality of fluid conduits (e.g., microchannels, micro-reservoirs, library storage elements, etc.). Alternatively, the voltage controller is electrically connected to less than all of the device's fluid conduits. In one embodiment, multiple electrodes are positioned to provide for switching of the electric field direction in the, e.g., microchannel(s), thereby causing the analytes to travel a longer distance than the physical length of the microchannel. Use of electrokinetic transport to control material movement in interconnected channel structures is described in, e.g., WO 96/94547 to Ramsey. An exemplary controller is described in U.S. Pat. No. 5,800,690. Modulating voltages are concomitantly applied to the various fluid areas of the device to affect a desired fluid flow characteristic, e.g., continuous or discontinuous (e.g., a regularly pulsed field causing the sample to oscillate direction of travel) flow of labeled components toward a waste reservoir. Particularly, modulation of the voltages applied at the various areas can move and direct fluid flow through the interconnected channel structure of the device.

The controlling instrumentation discussed above is also optionally used to provide for electrokinetic injection or withdrawal of material downstream of a region of interest to control an upstream flow rate. The same instrumentation and techniques described above are also utilized to inject a fluid into a downstream port to function as a flow control element.

The current invention also optionally includes other methods of fluid transport, e.g., available for situations in which electrokinetic methods are not desirable. For example, fluid transport and direction, sample reconstitution and reaction, etc. are optionally carried out in whole, or in part, in a pressure-based system to, e.g., avoid electrokinetic biasing during sample mixing. High throughput systems typically use pressure induced sample introduction. Pressure based flow is also desirable in systems in which electrokinetic transport is also used. For example, pressure based flow is optionally used for introducing and reacting reagents in a system in which the products are electrophoretically separated. In the present invention molecules are optionally loaded and other reagents are flowed through the microchannels or micro-reservoirs using, e.g., electrokinetic fluid control and/or under pressure.

Pressure is optionally applied to the microscale elements of the invention, e.g., to a microchannel, micro-reservoir, library storage element, region, etc. to achieve fluid movement using any of a variety of techniques. Fluid flow and flow of materials suspended or solubilized within the fluid, including cells or molecules, is optionally regulated by pressure based mechanisms such as those based upon fluid displacement, e.g., using a piston, pressure diaphragm, vacuum pump, probe, or the like, to displace liquid and/or gas and raise or lower the pressure at a site in the microfluidic system. The pressure is optionally pneumatic, e.g., a pressurized gas, or uses hydraulic forces, e.g., pressurized liquid, or alternatively, uses a positive displacement mechanism, e.g., a plunger fitted into a material reservoir, for forcing material through a channel or other conduit, or is a combination of such forces. Internal sources include microfabricated pumps, e.g., diaphragm pumps, thermal pumps, lamb wave pumps and the like that have been described in the art. See, e.g., U.S. Pat. Nos. 5,271,724; 5,277,566; and 5,375,979 and Published PCT Application Nos. WO 94/05414 and WO 97/02347.

In some embodiments, a pressure source is applied to a reservoir or well at one end of a microchannel to force a fluidic material through the channel. Optionally, the pressure can be applied to multiple ports at channel termini, or, a single pressure source can be used at a main channel terminus. Optionally, the pressure source is a vacuum source applied at the downstream terminus of the main channel or at the termini of multiple channels. Pressure or vacuum sources are optionally supplied externally to the device or system, e.g., external vacuum or pressure pumps sealably fitted to the inlet or outlet of channels or to the surface openings of micro-reservoirs, or they are internal to the device, e.g., microfabricated pumps integrated into the device and operably linked to channels or they are both external and internal to the device. Examples of microfabricated pumps have been widely described in the art. See, e.g., published International Application No. WO 97/02357.

These applied pressures or vacuums generate pressure differentials across the lengths of channels to drive fluid flow through them. In the interconnected channel networks described herein, differential flow rates on volumes are optionally accomplished by applying different pressures or vacuums at multiple ports, or, by applying a single vacuum at a common waste port and configuring the various channels with appropriate resistance to yield desired flow rates. In the present invention, for example, vacuum/pressure sources optionally apply different pressure levels to various channels to switch flow between the channels or to deliver flow to specific library storage elements. As discussed above, this is optionally done with multiple sources or by connecting a single source to a valve manifold comprising multiple electronically controlled valves, e.g., solenoid valves.

Hydrostatic, wicking and capillary forces are also optionally used to provide fluid flow of materials such as reconstituted library samples (or, alternatively to reconstitute the library samples), reagents, buffers, etc. in the invention. See, e.g., “METHOD AND APPARTUS FOR CONTINUOUS LIQUID FLOW IN MICROSCALE CHANNELS USING PRESSURE INJECTION, WICKING AND ELECTROKINETIC INJECTION,” by Alajoki et al., U.S. Ser. No. 09/245,627, filed Feb. 5, 1999. In using wicking/capillary methods, an adsorbent material or branched capillary structure is placed in fluidic contact with a region where pressure is applied, thereby causing fluid to move towards the adsorbent material or branched capillary structure. Furthermore, the capillary forces are optionally used in conjunction with electrokinetic or pressure-based flow in the channels, etc. of the present invention in order to pull material, etc. through the channels. Additionally, a wick is optionally added to draw fluid through a porous matrix fixed in a microscale channel or capillary.

Use of a hydrostatic pressure differential is another way to control flow rates through the channels, etc. of the present invention. For example, in a simple passive aspect, a cell suspension is deposited in a reservoir or well at one end of a channel at sufficient volume or liquid height so that the cell suspension creates a hydrostatic pressure differential along the length of the channel by virtue of, e.g., the cell suspension reservoir having greater liquid height than a well at an opposite terminus of the channel. Typically, the reservoir volume is quite large in comparison to the volume or flow-through rate of the channel, e.g., 10 microliter reservoirs or larger as compared to a 100 micrometer channel cross section.

The present invention optionally includes mechanisms for reducing adsorption of materials during fluid-based flow, e.g., as are described in U.S. Ser. No. 09/310,027 filed May 11, 1999 by Parce et al. In brief, adsorption of components, proteins, enzymes, markers and other materials to channel walls or other microscale components during pressure-based flow can be reduced by applying an electric field such as an alternating current to the material during flow. Alternatively, flow rate changes due to adsorption are detected and the flow rate is adjusted by a change in pressure or voltage.

The invention also optionally includes mechanisms for focusing labeling reagents, reconstituted library samples, enzymes, modulators, and other components into the center of microscale flow paths, which is useful in increasing assay throughput by regularizing flow velocity, e.g., in pressure based flow, e.g., as are described in U.S. Ser. No. 60/134,472 by H. Garrett Wada et al., filed May 17, 1999. In brief, sample materials are focused into the center of a channel by forcing fluid flow from opposing side channels into the main channel, or by other fluid manipulation.

In an alternate embodiment, microfluidic systems of the invention can be incorporated into centrifuge rotor devices, which are spun in a centrifuge. Fluids and particles thus travel through the device due to gravitational and centripetal/centrifugal pressure forces.

One use of an optional fluid control embodiment of the invention is illustrated by the following non-limiting example describing reconstitution of library samples from library storage elements. Those of skill in the art will readily recognize a variety of non-critical parameters that could be changed or modified to yield essentially similar or desirably different results.

In some embodiments of the invention, library samples are stored within open-well micro-reservoirs wherein the library sample is disposed within the micro-reservoir (as opposed to being within, e.g., a test-microchannel, etc.). One optional way to reconstitute such samples involves flowing a first fluid, e.g., a buffer, through the microchannel leading to the micro-reservoir. The fluid is stopped before entering the micro-reservoir itself. The fluid can be flowed through the microchannel by, e.g., any of the above described fluid control methods such as, e.g., pressure based flow, etc. For example, the flow of such first fluid can be driven by capillary force which will naturally stop when the fluid reaches the reservoir (i.e., when the fluid reaches the end of the microchannel). Vacuum can then be applied and the flow will not be reversed unless the vacuum is stronger than the capillary forces. Subsequently, a second fluid (comprising either the same type of fluid as the first sample or a different fluid type) can be optionally added into the open-well micro-reservoir onto the stored library sample. The addition is optionally done by hand (e.g., pipetted into configurations wherein the open-well micro-reservoir is large enough to allow such) or by, e.g., robotic means. After fluid is added into the open-well micro-reservoir, the addition of fluid to the reservoir will reduce the capillary force therein and flow will commence from the reservoir until the fluid/air interface reaches the entrance to the microchannel where the capillary force increases (i.e., the fluid will exit the reservoir). The fluid (containing the reconstituted sample) thus flows out of the micro-reservoir and into the rest of the microchannel array etc.

The conditions of fluid flow out of a micro-reservoir can be altered in numerous ways depending upon the specific need of the assay being used, etc. For example the size (e.g., volume, depth, etc.) of the open-well micro-reservoirs can be changed. A change in reservoir size can include, e.g., enlarging them enough so as to allow hand pipetting into them. Additionally, the reservoir size can be changed in order to change the time needed for reconstituted sample to flow out of the micro-reservoir. Larger reservoirs containing more fluid require longer times for fluids to empty out of them than do smaller reservoirs which contain less fluid (compared when going into the same size microchannel). Conversely, smaller reservoir sizes require less time to empty out into the same size microchannel. The sizes of, e.g., both the micro-reservoirs and the microchannels into which the micro-reservoirs drain can be changed in order to change the time required to flow out a reconstituted library sample. In various embodiments these parameters are changed, depending upon the specific needs/parameters of the samples, assays, etc. being used. Of course, in addition to, or alternatively to, the just described method, the reconstituted library samples (and the reconstitution of the library samples) can be done using other flow techniques, e.g., such as those described, supra, e.g., pressure based flow, etc.

In other embodiments of the invention, library samples are deposited within test-microchannels which are connected to open-well micro-reservoirs. The control of fluid flow to and from such test-microchannels can be controlled in similar fashion as to the above example. However, since the library sample is deposited within the test-microchannel instead of within the micro-reservoir, the sample becomes reconstituted when fluid is flowed into the test-microchannel. This is as opposed to the sample becoming reconstituted when fluid enters the micro-reservoir as occurs in the previous example. Again, in reference to the above example, here, the reconstituted library sample would flow out of the test-microchannel when a fluidic material is added to the connected micro-reservoir. Again, the fluid flow to and from the library storage element (when such is a test-microchannel) can be by any fluid flow means, e.g., as described herein (or a combination of such means) such as hydrostatic, pressure, etc.

Fluid flow or particle flow in the present devices and methods is optionally achieved using any one or more of the above techniques, alone or in combination. Typically, the controller systems involved are appropriately configured to receive or interface with a microfluidic device or system element as described herein. For example, the controller, optionally includes a stage upon which the device of the invention is mounted to facilitate appropriate interfacing between the controller and the device. Typically, the stage includes an appropriate mounting/alignment structural element, such as a nesting well, alignment pins and/or holes, asymmetric edge structures (to facilitate proper device alignment), and the like. Many such configurations are described in the references cited herein.

Detection

In general, detection systems in microfluidic devices include, e.g., optical sensors, temperature sensors, pressure sensors, pH sensors, conductivity sensors, and the like. Each of these types of sensors is readily incorporated into the microfluidic systems described herein. In these systems, such detectors are placed either within or adjacent to the microfluidic device or one or more microchannels, microchambers, micro-reservoirs, library storage elements or conduits of the device, such that the detector is within sensory communication with the device, channel, reservoir, or chamber, etc. The phrase “proximal,” to a particular element or region, as used herein, generally refers to the placement of the detector in a position such that the detector is capable of detecting the property of the microfluidic device, a portion of the microfluidic device, or the contents of a portion of the microfluidic device, for which that detector was intended. For example, a pH sensor placed in sensory communication with a microscale channel is capable of determining the pH of a fluid disposed in that channel. Similarly, a temperature sensor placed in sensory communication with the body of a microfluidic device is capable of determining the temperature of the device itself.

Many different molecular/reaction characteristics can be detected in microfluidic devices of the current invention. For example, various embodiments can detect such things as fluorescence or emitted light, changes in the thermal parameters (e.g., heat capacity, etc.) involved in the assays, etc.

Examples of detection systems in the current invention can include, e.g., optical detection systems for detecting an optical property of a material within, e.g., the microchannels of the microfluidic devices that are incorporated into the microfluidic systems described herein. Such optical detection systems are typically placed adjacent to a microscale channel of a microfluidic device, and optionally are in sensory communication with the channel via an optical detection window or zone that is disposed across the channel or chamber of the device.

Optical detection systems of the invention include, e.g., systems that are capable of measuring the light emitted from material within the channel, the transmissivity or absorbance of the material, as well as the material's spectral characteristics, e.g., fluorescence, chemiluminescence. Detectors optionally detect a labeled compound, such as fluorographic, colorimetric or radioactive component. Types of detectors optionally include spectrophotometers, photodiodes, avalanche photodiodes, microscopes, scintillation counters, cameras, diode arrays, imaging systems, photomultiplier tubes, CCD arrays, scanning detectors, galvo-scanners, film and the like, as well as combinations thereof. Proteins, antibodies, or other components which emit a detectable signal can be flowed past the detector, or alternatively, the detector can move relative to an array to determine, e.g., molecule position (or, the detector can simultaneously monitor a number 5 of spatial positions corresponding to channel regions, e.g., as in a CCD array). Examples of suitable detectors are widely available from a variety of commercial sources known to persons of skill. See, also, The Photonics Design and Application Handbook, books 1, 2, 3 and 4, published annually by Laurin Publishing Co., Berkshire Common, P.O. Box 1146, Pittsfield, Mass. for common sources for optical components.

As noted above, the present devices optionally include, as microfluidic devices typically do, a detection window or zone at which a signal, e.g., fluorescence, is monitored. This detection window or zone optionally includes a transparent cover allowing visual or optical observation and detection of the, e.g., assay results, e.g., observation of a colorimetric, fluorometric or radioactive response, or a change in the velocity of calorimetric, fluorometric or radioactive component.

Another optional embodiment of the present invention involves use of fluorescence correlation spectroscopy and/or confocal nanofluorimetric techniques to detect fluorescence from the molecules in the microfluidic device. Such techniques are easily available (e.g., from Evotec, Hamburg, Germany) and involve detection of fluorescence from molecules that diffuse through the illuminated focus area of a confocal lens. The length of any photon burst observed will correspond to the time spent in the confocal focus by the molecule. The diffusion coefficient of the molecules passing through this area can be used to measure, e.g., degree of binding between different library samples or between samples from different libraries. Various algorithms used for analysis can be used to evaluate fluorescence signals from individual molecules based on changes in, e.g., brightness, fluorescence lifetime, spectral shift, FRET, quenching characteristics, etc.

The sensor or detection portion of the devices and methods of the present invention can optionally comprise a number of different apparatuses. For example, fluorescence can be detected by, e.g., a photomultiplier tube, a charge coupled device (CCD) (or a CCD camera), a photodiode, or the like.

A photomultiplier tube is an optional aspect of the current invention. Photomultiplier tubes (PMTs) are devices which convert light (photons) into electronic signals. The detection of each photon by the PMT is amplified into a larger and more easily measurable pulse of electrons. PMTs are commonly used in many laboratory applications and settings and are well known to those in the art.

Another optional embodiment of the present invention comprises a charge coupled device. CCD cameras can detect even very small amounts of electromagnetic energy (e.g., such that emitted by fluorophores in the present invention). CCD cameras are made from semiconducting silicon wafers that release free electrons when light photons strike the wafers. The output of electrons is linearly directly proportional to the amount of photons that strike the wafer. This allows the correlation between the image brightness and the actual brightness of the event observed. CCD cameras are very well suited for imaging of fluorescence emissions since they can detect even extremely faint events, can work over a broad range of spectrum, and can detect both very bright and very weak events. CCD cameras are well know to those in the art and several suitable examples include those made by: Stratagene (La Jolla, Calif.), Alpha-Innotech (San Leandro, Calif.), and Apogee Instruments (Tucson, Ariz.) among others.

Yet another optional embodiment of the present invention comprises use of a photodiode to detect fluorescence from the molecules in the microfluidic device. Photodiodes absorb incident photons which cause electrons in the photodiode to diffuse across a region in the diode thus causing a measurable potential difference across the device. This potential can be measured and is directly related to the intensity of the incident light.

In some aspects, the detector measures an amount of light emitted from the material, such as a fluorescent or chemiluminescent material. As such, the detection system will typically include collection optics for gathering a light based signal transmitted through the detection window or zone, and transmitting that signal to an appropriate light detector. Microscope objectives of varying power, field diameter, and focal length are readily utilized as at least a portion of this optical train. The detection system is typically coupled to a computer (described in greater detail below), via an analog to digital or digital to analog converter, for transmitting detected light data to the computer for analysis, storage and data manipulation.

In the case of fluorescent materials such as labeled cells or fluorescence indicator dyes or molecules, the detector and/or detection system optionally includes a light source which produces light at an appropriate wavelength for activating the fluorescent material, as well as optics for directing the light source to the material contained in the channel. The light source can be any number of light sources that provides an appropriate wavelength, including, e.g., lasers, laser diodes and LEDs. Other light sources are optionally utilized for other detection systems. For example, broad band light sources for light scattering/transmissivity detection schemes, and the like. Typically, light selection parameters are well known to those of skill in the art.

The detector can exist as a separate unit, but is preferably integrated with the controller system, into a single instrument. Integration of these functions into a single unit facilitates connection of these instruments with a computer (described below), by permitting the use of few or a single communication port(s) for transmitting information between the controller, the detector and the computer. Integration of the detection system with a computer system typically includes software for converting detector signal information into assay result information, e.g., concentration of a substrate, concentration of a product, presence of a compound of interest, interaction between various library samples, or the like.

Computer

As noted above, either, or both, the fluid direction system or the detection system as well as other aspects of the current invention described herein (e.g., temperature control, etc.) are optionally coupled to an appropriately programmed processor or computer that functions to instruct the operation of these instruments in accordance with preprogrammed or user input instructions, receive data and information from these instruments, and interpret, manipulate and report this information to the user. As such, the computer is typically appropriately coupled to one or more of the appropriate instruments (e.g., including an analog to digital or digital to analog converter as needed).

The computer optionally includes appropriate software for receiving user instructions, either in the form of user input into set parameter fields, e.g., in a GUI, or in the form of preprogrammed instructions, e.g., preprogrammed for a variety of different specific operations. The software then converts these instructions to appropriate language for instructing the operation of, e.g., the fluid direction and transport controller to carry out the desired operation.

For example, the computer is optionally used to direct a fluid direction system to control fluid flow, e.g., into and through a variety of interconnected microchannels. The fluid direction system optionally directs the movement of, e.g., fluid flow to and from the various library storage elements of the invention (e.g., for reconstitution of the contained library samples). Additionally, the fluid direction system optionally directs fluid flow controlling which reconstituted library samples are contacted with each other and/or with various reagents, buffers, etc. in, e.g., a detection region or other region(s) in the microfluidic device. Furthermore, the fluid direction system optionally controls the coordination of movements of multiple fluids/molecules/etc. concurrently as well as sequentially. For example, the computer optionally directs the fluid direction system to direct the movement of at least a first member of a plurality of molecules into a first member of a plurality of microchannels concurrent with directing the movement of at least a second member of the plurality of molecules into one or more detection channel regions. Additionally or alternatively, the fluid direction system directs the movement of at least a first member of the plurality of molecules into the plurality of microchannels concurrent with incubating at least a second member of the plurality of molecules or directs movement of at least a first member of the plurality of molecules into the one or more detection channel regions concurrent with incubating at least a second member of the plurality of molecules.

By coordinating channel switching, the computer controlled fluid direction system directs the movement of at least one member of the plurality of molecules into the plurality of microchannels and/or one member into a detection region at a desired time interval, e.g., greater than 1 minute, about every 60 seconds or less, about every 30 seconds or less, about every 10 seconds or less, about every 1.0 seconds or less, or about every 0.1 seconds or less. Each sample, with appropriate channel switching as described above, remains in the plurality of channels for a desired period of time, e.g., between about 0.1 minutes or less and about 60 minutes or more. For example the samples optionally remain in the channels for a selected incubation time of, e.g., 20 minutes.

The computer then optionally receives the data from the one or more sensors/detectors included within the system, interprets the data, and either provides it in a user understood format, or uses that data to initiate further controller instructions, in accordance with the programming, e.g., such as in monitoring and control of flow rates (e.g., as involved in reconstitution of specific library samples, etc.), temperatures, applied voltages, pressures, and the like.

In the present invention, the computer typically includes software for the monitoring and control of materials in the various microchannels, etc. For example, the software directs channel switching to control and direct flow as described above. Additionally the software is optionally used to control electrokinetic, pressure-modulated, or the like, injection or withdrawal of material. The injection or withdrawal is used to modulate the flow rate as described above. The computer also typically provides instructions, e.g., to the controller or fluid direction system for switching flow between channels to achieve a high throughput format.

In addition, the computer optionally includes software for deconvolution of the signal or signals from the detection system. For example, the deconvolution distinguishes between two detectably different spectral characteristics that were both detected, e.g., when a substrate and product comprise detectably different labels.

Any controller or computer optionally includes a monitor which is often a cathode ray tube (CRT) display, a flat panel display (e.g., active matrix liquid crystal display, liquid crystal display), or the like. Data produced from the microfluidic device, e.g., fluorographic indication of binding between selected molecules, is optionally displayed in electronic form on the monitor. Additionally, the data gathered from the microfluidic device can be outputted in printed form. The data, whether in printed form or electronic form (e.g., as displayed on a monitor), can be in various or multiple formats, e.g., curves, histograms, numeric series, tables, graphs and the like.

Computer circuitry is often placed in a box which includes, e.g., numerous integrated circuit chips, such as a microprocessor, memory, interface circuits, etc. The box also optionally includes such things as a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

Example Integrated System

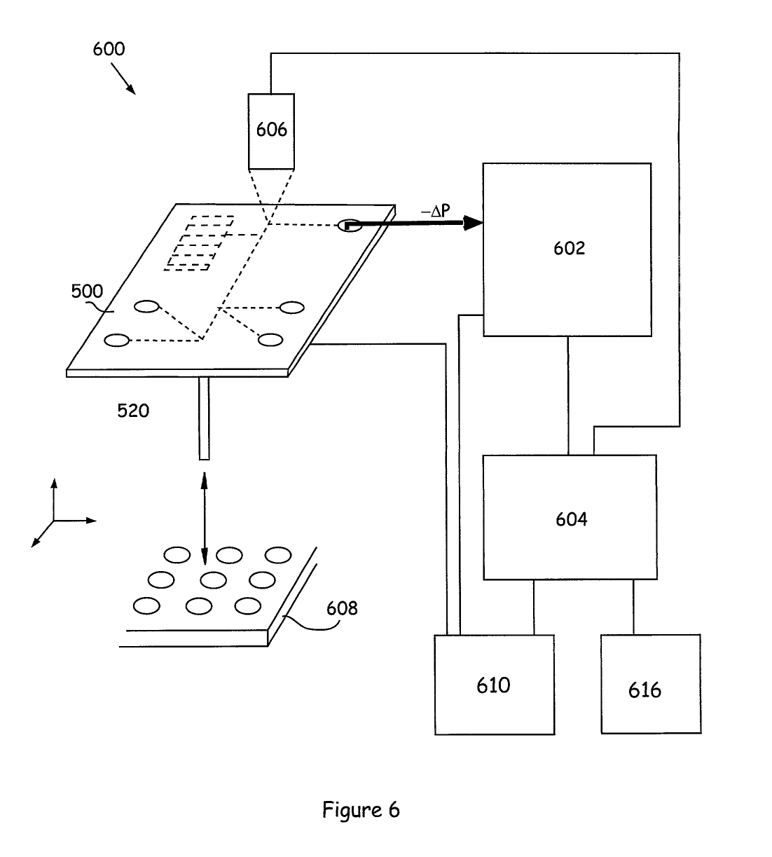

FIG. 5, Panels A, B, and C and FIG. 6 provide additional details regarding example integrated systems that optionally use the devices of the invention and optionally are used to practice the methods herein. As shown in FIG. 5, body structure 502 has main channel 504 disposed therein. A sample or mixture of components, e.g., typically a buffer, is optionally flowed from pipettor channel 520 towards reservoir 514, e.g., by applying a vacuum at reservoir 514 (or another point in the system) or by applying appropriate voltage gradients or wicking arrangements. Alternatively, a vacuum, or appropriate pressure force, is applied at, e.g., reservoirs 508, 512 or through pipettor channel 520. Optionally, integrated systems using the devices and methods of the invention do not utilize pipettor channels or the like. The microfluidic libraries of the invention with the plethora of library storage elements, etc. allow for assays, etc. wherein no outside reagents, etc. need to be drawn in through such pipettor channels, etc.

Additional materials, such as buffer solutions, substrate solutions, enzyme solutions, test molecules, fluorescence indicator dyes or molecules and the like, as described herein, are optionally flowed from wells, e.g., 508 or 512 and into main channel 504. Flow of, e.g., buffer, etc. also optionally travels from the main channel, 504, to, e.g., open-well micro-reservoir 530 (i.e., a library storage element) in library array 528 where library samples are reconstituted. In this example the library storage element is contained within an open-well micro-reservoir, but such could also contained within a test-microchannel, etc. In preferred embodiments, library arrays of the invention comprise between 5 and 10,000 or more library storage elements per square centimeter. Additionally, and optionally, other fluidic reagents, buffers, etc. can be admitted into library storage elements that comprise open-well micro-reservoirs, e.g., open-well micro-reservoir 530. Flow from the micro-reservoir 530 is optionally performed, e.g., by modulating fluid pressure, by electrokinetic approaches, by wicking forces, by hydrostatic forces, etc. as described, supra, (or a combination of such forces, etc.). As fluid is added to main channel 504, e.g., from reservoir 508, the flow rate increases. The flow rate is optionally reduced by flowing a portion of the fluid from main channel 504 into flow reduction channel 506 or 510. The arrangement of channels depicted in FIG. 5 is only one possible arrangement out of many which are appropriate and available for use in the present invention. Additional alternatives can be readily devised, e.g., by combining the microfluidic elements described herein, e.g., flow reduction channels, with other microfluidic devices described in the patents and applications referenced herein. Also, as described previously, optional embodiments of the invention can include, e.g., multiple libraries on the same microfluidic device, alternative configurations of microchannels (e.g., microchannel 532) leading to library storage elements, variation in size and number of library storage elements, configuration of library arrays, etc.

Samples and materials are optionally flowed from the enumerated wells or from a source external to the body structure or, more preferably, from a library storage element (e.g., micro-reservoir 530). As depicted, the integrated system optionally includes pipettor channel 520, e.g., protruding from body 502, for accessing a source of materials external to the microfluidic system. Typically, the external source is a microtiter dish or other convenient storage medium. For example, as depicted in FIG. 6, pipettor channel 520 can access microwell plate 608, which optionally includes, e.g., reconstitution buffers, fluorescence dyes, various fluidic reagents to be interacted with the library samples contained within the library arrays, etc., in the wells of the plate. Again, however, the methods and devices of the current invention easily allow for use wherein no outside storage areas (e.g., microwell plates, etc.) or pipettor capillaries are involved. In fact, typical applications of the invention need not use either pipettor capillaries or external storage areas such as microwell plates.

Detector 606 is in sensory communication with channel 504, detecting signals resulting, e.g., from labeled materials flowing through the detection region, changes in heat capacity or other thermal parameters, fluorescence, etc. Detector 606 is optionally coupled to any of the channels or regions of the device where detection is desired. Detector 606 is operably linked to computer 604, which digitizes, stores, and manipulates signal information detected by detector 606, e.g., using any of the instructions described above or any other instruction set, e.g., for determining concentration, molecular weight or identity, interaction between library samples and test molecules, or the like.

Fluid direction system 602 controls voltage, pressure, etc. (or a combination of such), e.g., at the wells of the systems or through the channels of the system, or at vacuum couplings fluidly coupled to channel 504 or other channel described above. Optionally, as depicted, computer 604 controls fluid direction system 602. In one set of embodiments, computer 604 uses signal information to select further parameters for the microfluidic system. For example, upon detecting the interaction between a particular library sample and a first reagent, the computer optionally directs addition of a second reagent of interest into the system to be tested against that particular library sample.

Temperature control system 610 controls joule and/or non-joule heating at the wells of the systems or through the channels of the system as described herein. Optionally, as depicted, computer 604 controls temperature control system 610. In one set of embodiments, computer 604 uses signal information to select further parameters for the microfluidic system. For example, upon detecting the desired temperature in a sample in channel 504, the computer optionally directs addition of, e.g., a potential binding molecule, fluorescence indicator dye, etc. into the system to be tested against one or more library samples.

Monitor 616 displays the data produced by the microfluidic device, e.g., graphical representation of interaction (if any) between each library sample and a series of reagents, test molecules, etc. Optionally, as depicted, computer 604 controls monitor 616. Additionally, computer 604 is connected to and directs additional components such as printers, electronic data storage devices and the like.

Assay Kits

The present invention also provides kits for utilizing the library(ies) of the invention. In particular, these kits typically include microfluidic devices, systems, modules and workstations for utilizing the library(ies) of the invention. A kit optionally contains additional components for the assembly and/or operation of a multimodule workstation of the invention including, but not restricted to robotic elements (e.g., a track robot, a robotic armature, or the like), plate handling devices, fluid handling devices, and computers (including e.g., input devices, monitors, c.p.u., and the like).

Generally, the microfluidic devices described herein are optionally packaged to include some or all reagents for performing the device's functions in addition to the various library samples. For example, the kits can optionally include any of the microfluidic devices described along with assay components, buffers, reagents, enzymes, serum proteins, receptors, sample materials, antibodies, substrates, control material, spacers, buffers, immiscible fluids, etc., for performing the assays utilizing the methods and devices of the invention. In the case of prepackaged reagents, the kits optionally include pre-measured or pre-dosed reagents that are ready to incorporate into the assay methods without measurement, e.g., pre-measured fluid aliquots used to reconstitute the library components, or pre-weighed or pre-measured solid reagents that can be easily reconstituted by the end-user of the kit.

Such kits also typically include appropriate instructions for using the devices and reagents, and in cases where reagents (or all necessary reagents) are not predisposed in the devices themselves (e.g., as library samples), with appropriate instructions for introducing the reagents into the channels/chambers/reservoirs/etc. of the device. In this latter case, these kits optionally include special ancillary devices for introducing materials into the microfluidic systems, e.g., appropriately configured syringes/pumps, or the like (in one embodiment, the device itself comprises a pipettor element, such as an electropipettor for introducing material into channels/chambers/reservoirs/etc. within the device). In the former case, such kits typically include a microfluidic device with necessary reagents predisposed in the channels/chambers/reservoirs/etc. of the device. Generally, such reagents are provided in a stabilized form, so as to prevent degradation or other loss during prolonged storage, e.g., from leakage. A number of stabilizing processes are widely used for reagents that are to be stored, such as the inclusion of chemical stabilizers (i.e., enzymatic inhibitors, microbicides/bacteriostats, anticoagulants), the physical stabilization of the material, e.g., through immobilization on a solid support, entrapment in a matrix (i.e., a bead, a gel, etc.), lyophilization, or the like.

The elements of the kits of the present invention are typically packaged together in a single package or set of related packages. The package optionally includes written instructions for utilizing one or more library of the invention in accordance with the methods described herein. Kits also optionally include packaging materials or containers for holding the microfluidic device, system or reagent elements.

The discussion above is generally applicable to the aspects and embodiments of the invention described herein. Moreover, modifications are optionally made to the methods and devices described herein without departing from the spirit and scope of the invention as claimed, and the invention is optionally put to a number of different uses including the following:

The use of a microfluidic system containing at least a first substrate and having a first channel and a second channel intersecting the first channel, at least one of the channels having at least one cross-sectional dimension in a range from 0.1 to 500 micrometer, in order to test the effect of each of a plurality of test compounds on a biochemical system comprising one or more focused cells or particles.

The use of a microfluidic system as described herein, wherein a biochemical system flows through one of said channels substantially continuously, providing for, e.g., sequential testing of a plurality of test compounds.

The use of a microfluidic device as described herein to modulate reactions within microchannels/microchambers/reservoirs/etc.

The use of electrokinetic injection in a microfluidic device as described herein to modulate or achieve flow in the channels.

The use of a combination of wicks, electrokinetic injection and pressure based flow elements in a microfluidic device as described herein to modulate, focus, or achieve flow of materials, e.g., in the channels of the device.

An assay utilizing a use of any one of the microfluidic systems or substrates described herein.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A microfluidic device comprising:
(i) a body structure;
(ii) a plurality of dried or immobilized library storage elements located on or within the body structure; and,
(ii) a plurality of microscale channels located within the body structure, at least one of the plurality of microscale channels being fluidly coupled to the plurality of library storage elements.

2. The microfluidic device of claim 1, wherein the library storage elements are contained within one or more microscale reservoirs.

3. The microfluidic device of claim 2, wherein the one or more microscale reservoirs comprise a largest dimension, which largest dimension is less than about 5 mm.

4. The microfluidic device of claim 3, wherein the largest dimension is less than about 1 mm.

5. The microfluidic device of claim 4, wherein the largest dimension is less than about 500 μm.

6. The microfluidic device of claim 5, wherein the largest dimension is about 300 μm or less.

7. The microfluidic device of claim 2, wherein the one or more microscale reservoirs is disposed within a surface of the body structure of the microfluidic device.

8. The microfluidic device of claim 7, wherein the one or more microscale reservoirs is disposed within an upper surface of body structure of the microfluidic device.

9. The microfluidic device of claim 1, wherein the plurality of library storage elements comprises at least about 10 to about 1,000,000 or more library storage elements.

10. The microfluidic device of claim 9, wherein the plurality of library storage elements comprises at least about 100 to about 100,000 or more library storage elements.

11. The microfluidic device of claim 10, wherein the plurality of library storage elements comprises at least about 1,000 to about 10,000 or more library storage elements.

12. The microfluidic device of claim 1, wherein the plurality of library storage elements comprises at least about 60,000 to about 600,000 or more library storage elements.

13. The microfluidic device of claim 1, wherein the plurality of library storage elements comprises a density of at least about 5 to about 10,000 or more library storage elements per square centimeter of the body structure.

14. The microfluidic device of claim 13, wherein the plurality of library storage elements comprises a density of at least about 100 to about 5,000 or more library storage elements per square centimeter.

15. The microfluidic device of claim 14, wherein the plurality of library storage elements comprises a density of at least about 1,000 to about 2,500 or more library storage elements per square centimeter.

16. The microfluidic device of claim 13, wherein the plurality of library storage elements comprises a density of at least about 100 to about 500 or more library storage elements per square centimeter.

17. The microfluidic device of claim 13, wherein the plurality of library storage elements comprises a density of at least about 400 to about 4,000 or more library storage elements per square centimeter.

18. The microfluidic device of claim 1, wherein at least one member of the plurality of library storage elements comprises a dried or immobilized test compound.

19. The microfluidic device of claim 1, wherein substantially all members of the plurality of library storage elements comprise a different dried or immobilized test compound.

20. The microfluidic device of claim 1, wherein the plurality of library storage elements comprises a library of test compounds.

21. The microfluidic device of claim 1, wherein at least one member of the plurality of microscale channels includes a fluidic material contained within the microscale channel.

22. The microfluidic device of claim 21, wherein substantially all members of the plurality of microscale channels comprise a fluidic material contained within the microscale channels.

23. The microfluidic device of claim 21, wherein the fluidic material comprises a buffer.

24. The device of claim 1, wherein at least one member of the plurality of library storage elements comprises a dried or immobilized test compound and at least one member of the plurality of microscale channels comprises a fluidic material, which fluidic material contacts the dried or immobilized test compound.

25. The device of claim 1, wherein at least one member of the plurality of library storage elements comprises a dried or immobilized test compound and at least one member of the plurality of microscale channels comprises a fluidic material, which fluidic material contacts the dried or immobilized test compound, which compound has been reconstituted by at least a second fluidic material.

26. A microfluidic system, the system comprising:
(i) a body structure having a plurality of microscale channels disposed therein and a plurality of dried or immobilized library storage elements disposed on or within the body structure, at least one of the microscale channels being fluidly connected to the plurality of library storage elements; and,
(ii) a fluid delivery system operable to deliver at least a first fluid to at least one or more member of the plurality of library storage elements.

27. The microfluidic system of claim 26, wherein the library storage elements are contained within one or more microscale reservoirs.

28. The microfluidic system of claim 27, wherein the microscale reservoirs comprise a largest dimension, which largest dimension is less than about 5 mm.

29. The microfluidic system of claim 28, wherein the largest dimension is less than about 1 mm.

30. The microfluidic system of claim 29, wherein the largest dimension is less than about 500 μm.

31. The microfluidic system of claim 30, wherein the largest dimension is about 300 μm or less.

32. The microfluidic system of claim 27, wherein the plurality of microscale reservoirs is disposed within a surface of the body structure.

33. The microfluidic system of claim 32, wherein the plurality of microscale reservoirs is disposed within an upper surface of the body structure.

34. The microfluidic system of claim 26, wherein the plurality of library storage elements comprises at least about 10 to about 1,000,000 or more library storage elements.

35. The microfluidic system of claim 34, wherein the plurality of library storage elements comprises at least about 100 to about 100,000 or more library storage elements.

36. The microfluidic system of claim 35, wherein the plurality of library storage elements comprises at least about 1,000 to about 10,000 or more library storage elements.

37. The microfluidic system of claim 34, wherein the plurality of library storage elements comprises at least about 60,000 to about 600,000 or more library storage elements.

38. The microfluidic system of claim 26, wherein the plurality of library storage elements comprises a density of about 5 to about 10,000 or more library storage elements per square centimeter of the body structure

39. The microfluidic system of claim 38, wherein the plurality of library storage elements comprises a density of at least about 100 to about 5,000 or more library storage elements per square centimeter.

40. The microfluidic system of claim 39, wherein the plurality of library storage elements comprises a density of at least about 1,000 to about 2,500 or more library storage elements per square centimeter.

41. The micro fluidic system of claim 39, wherein the plurality of library storage elements comprises a density of at least about 100 to about 500 or more library storage elements per square centimeter.

42. The microfluidic system of claim 38, wherein the plurality of library storage elements comprises a density of at least about 400 to about 4,000 or more library storage elements per square centimeter.

43. The microfluidic system of claim 26, wherein at l east one member of the plurality of library storage elements comprises a dried or immobilized test compound.

44. The microfluidic system of claim 26, wherein substantially all members of the plurality of library storage elements comprise a different dried or immobilized test compound.

45. The microfluidic system of claim 26, wherein the plurality of library storage elements comprises a library of test compounds.

46. The microfluidic system of claim 26, wherein at least one member of the plurality of microscale channels contains a fluidic material disposed therein.

47. The microfluidic system of claim 46, wherein substantially all members of the plurality of microscale channels contains a fluidic material disposed therein.

48. The microfluidic system of claim 46, wherein the fluidic material comprises a buffer material.

49. The microfluidic system of claim 26, wherein the fluid delivery system includes a pipettor device.

50. The microfluidic system of claim 26, wherein the fluid comprises a buffer material.

51. The microfluidic system of claim 26, wherein the fluid comprises less than about 20 microliters.

52. The microfluidic system of claim 51, wherein the fluid comprises less than about 5 microliters.

53. The microfluidic system of claim 52, wherein the fluid comprises less than about 1 microliter.

54. The microfluidic system of claim 53, wherein the fluid comprises less than about 200 nanoliters.

55. The microfluidic system of claim 54, wherein the fluid comprises less than about 50 nanoliters.

56. The microfluidic system of claim 55, wherein the fluid comprises less than about 10 nanoliters.

57. The microfluidic system of claim 56, wherein the fluid comprises less than about 2 nanoliters.

58. The microfluidic system of claim 57, wherein the fluid comprises about 1 nanoliter or less.

59. The microfluidic system of claim 26, wherein the fluid delivery system simultaneously delivers the fluid to at least about 2 to about 1,000,000 or more library storage elements.

60. The microfluidic system of claim 59, wherein the fluid is simultaneously delivered to at least about 5 or more library storage elements.

61. The microfluidic system of claim 26, wherein the fluid delivery system delivers the fluid to one or more member of the plurality of library storage elements about every 1 minute or less.

62. The microfluidic system of claim 61, wherein the fluid is delivered about every 30 seconds or less.

63. The microfluidic system of claim 65, wherein the fluid is delivered about every 10 seconds or less.

64. The microfluidic system of claim 66, wherein the fluid is delivered about every 5 seconds or less.

65. The microfluidic system of claim 67, wherein the fluid is delivered about every 1 second or less.

66. The microfluidic system of claim 27, wherein the fluid direction system directs:
(i) movement of a first fluidic material through at least a first microscale channel of the plurality of microscale channels to at least a first microscale reservoir which is fluidly connected to at least the first microscale channel; and
(ii) delivery of a second fluidic material to the first microscale reservoir.

67. The microfluidic system of claim 66 wherein the first fluidic material is directed to contact at least one dried or immobilized test compound disposed within the first microscale reservoir.

68. The microfluidic system of claim 67 wherein at least one of the first and second fluidic materials reconstitutes the at least one dried or immobilized test compound.

69. A method of loading at least a first test compound located within at least a first one of a plurality of microscale reservoirs into a microchannel system, which plurality of microscale reservoirs is fluidly coupled to the microchannel system, the method comprising:
(i) providing the at least first test compound in a dried or immobilized format in the at least first one of the plurality of microscale reservoirs;
(ii) flowing at least a first fluidic material into the at least first microscale reservoir; and
(iii) flowing the first fluidic material from at least the first microscale reservoir through at least a first microchannel into the microchannel system, thereby loading at least the first test compound into the microchannel system.

70. The method of claim 69 wherein the first fluidic material contacts the at least first test compound disposed within the first microscale reservoir.

71. The method of claim 70 further comprising delivering a second fluidic material into at least the first microscale reservoir.

72. The method of claim 71 wherein at least one of the first or second fluidic material reconstitutes the at least first test compound to make the test compound flowable.

73. The method of claim 69 further comprising reconstituting the first test compound prior to said (ii) flowing step

74. A method of loading at least a first test compound from at least a first one of a plurality of microscale test channels into a microchannel system, which plurality of microscale test channels is fluidly coupled to the microchannel system, the method comprising:
(i) providing the at least first test compound in a dried or immobilized format within the at least first one of the plurality of microscale test channels;
(ii) flowing at least a first fluidic material into the at least first microscale test channel; and
(iii) flowing the first fluidic material from at least the first microscale test channel through at least a first microchannel into the microchannel system, thereby loading at least the first test compound into the microchannel system.

75. The method of claim 74 wherein the first fluidic material contacts the at least first test compound disposed within the first microscale test channel.

76. The method of claim 75 further comprising delivering a second fluidic material into at least the first microscale test channel.

77. The method of claim 76 wherein at least one of the first or second fluidic material reconstitutes the at least first test compound to make the test compound flowable.

78. The method of claim 74 further comprising reconstituting the first test compound prior to said (ii) flowing step

79. The method of claims 71 or 76 further comprising delivering the second fluidic material into at least the first microscale reservoir or test channel by hand pipetting or by robotic pipetting.

80. The method of claim 69, wherein the (ii) and (iii) flowing steps comprise flowing the first fluidic material electrokinetically or by pressure-based flow.

81 The method of claims 69 or 74 wherein the first fluidic material dissolves the first test compound.

82. The method of claims 71 or 76 wherein the second fluidic material dissolves the first test compound.

83. The method of claim 71 wherein the first fluidic material and the second fluidic material comprise the same material.

84. The method of claim 83, wherein the first fluidic material and the second fluidic material each comprise a buffer material.

85. The method of claims 69 or 74 further comprising repeating steps (i) through (iii) for at least one or more test compounds.

86. A microfluidic device comprising:
(i) a body structure;
(ii) a plurality of dried or immobilized test compounds located on or within the body structure; and,
(ii) a plurality of microscale channels located within the body structure and fluidly coupled to the plurality of test compounds.
